(19) United States
(12) Patent Application Publication
(10) Pub. No.: US 2018/0374571 A1
(43) Pub. Date: Dec. 27, 2018

(54) CREDO Logging System

(71) Applicant: Edward Via College of Osteopathic Medicine, Blacksburg, VA (US)

(72) Inventors: Harold Garner, Blacksburg, VA (US); Fred Rawlins, Radford, VA (US)

(73) Assignee: Edward Via College of Osteopathic Medicine, Blacksburg, VA (US)

(21) Appl. No.: 16/015,735

(22) Filed: Jun. 22, 2018

Related U.S. Application Data
(60) Provisional application No. 62/523,822, filed on Jun. 23, 2017.

(51) Int. Cl. G16H 40/20; G16H 70/20; G16H 70/40; G06F 3/0482; G09B 7/06; G16H 10/60

(52) U.S. Cl. CPC G16H 40/20; G16H 70/20; G16H 10/60; G06F 3/0482; G09B 7/06; G16H 70/40

(57) ABSTRACT

A system implemented on a distributed computer network for capturing experiences of healthcare trainees. The system includes input devices having an interface for interacting with a user including an input screen having data input fields, selection fields and activation buttons and output screens. Also included are one or more back-end databases configured to store input data from the input devices and to provide output data to the input devices.

### RELATED APPLICATIONS

This application claims the benefit of U.S. Provisional Application No. 62/523,822 filed Jun. 23, 2017, and herein incorporated by reference.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

There have been many attempts, some successful and therefore sustained, to develop applications to enhance the practice of medicine for clinicians and their patients. Systems developed for procedural logging have also been developed, including, for example, automated trauma patient coding by physicians dating back to the 1990s. The systems have focused on narrow, specialized sets of codes and thus lacked wide applicability and universality. Some applications, apps, have been developed specifically to enhance medical training and capture the actions of trainees. These apps have focused on accessing a variety of medical reference materials, but have not been used for logging student activities. None of the above-mentioned systems employed universal codes to measure medical trainee clinical performance.

Current medical school curricula do not include or only touch upon billing, diagnostic or the procedural codes used in the clinic, so students, even in their clinical rotation years, are not effectively trained in the use of the codes that will be a significant part of their professional career as they begin their practice. Also, the exact breadth and depth of medical student training in their clinical rotation years is not quantified; often their experiences are captured in their loosely organized handwritten notes.

### BRIEF SUMMARY OF THE INVENTION

In one embodiment, the present invention addresses the above issues and others by providing a simple, easy to use “app” that, among other things, captures the diagnostic and procedural experiences of medical students via the internationally accepted ICD (International Statistical Classification of Diseases and Related Health Problems) system created and maintained by World Health Organization (WHO).

In other embodiments, the present invention provides an “app” to capture the clinical experiences of medical trainees and then use that information to improve clinical training of medical students, provide students with real-time feedback that they can use to enhance the depth and completeness of their training, and can be used by medical school administrators to quantify and document new accreditation requirements, identify students that are exceptional or at risk, and identify ways to improve and couple medical school learning, both basic and clinical that spans all years of medical school. As a result, better training will produce better doctors, thus improve health care quality nationally.

In other embodiments, the present invention uses Health Information Technology that captures the details of each user's clinical encounters via ICD diagnostic and procedural codes entered by trainees using electronic devices such as a mobile device. The information may be used to then dynamically enhance the training provided.

In other embodiments, the present invention uses a plurality of users at a plurality of locations involving a plurality of patient encounters and training sessions.

In a preferred embodiment, the present invention uses ICD codes to provide a common universal medical vocabulary which can capture diagnoses, procedures and drugs during patient encounters, and then use those codes to associate a variety of information (learning objectives, basic science facts, medical board review questions) to enhance student performance and evaluation.

In other embodiments, the present invention uses a spectrum of entries captured at various clinical training sites to evaluate each site and preceptor as to the quality, quantity, and variety of medical situations encountered by a user such as a student and then take action to ensure uniformity, quality, and adherence to accreditation requirements.

In other embodiments, the present invention can be used to significantly improve clinical training and thus better prepare health care workers for the reality of clinical situations.

In other embodiments, the present invention enables the sampling of public health status across a broad geographic region and across many healthcare institutions thereby presenting a new paradigm for public health monitoring with many ancillary benefits to the individuals and communities in a region.

In other embodiments, the present invention creates real-time interactive components that provide assessment information, and couple training opportunities (review facts and challenge questions) directly linked to the specific clinical patient encounters to enhance performance on medical board exams.

In other embodiments, the present invention is optimized for quick, easy and accurate entries, while being robust for a scale-up of users and translation to other medical schools.

In other embodiments, the present invention may be used to quantify and measure the uniformity of the clinical experiences across clinical rotation types, locales, and individual students to meet recent accreditation requirements.

In other embodiments, the present invention uses a native application to remove the need for continuous Internet access, thus enabling use in areas without cell phone or WIFI coverage that will sync periodically.

In other embodiments, the present invention enhances student and community public health awareness by developing analysis output, driven by student entries that provide actionable general health status for the rural socioeconomically diverse communities for a particular region.

In other embodiments, the present invention improves medicine by improving clinical training experiences. This may be done by providing an effective experience acquisition tool which provides valuable highly-relevant content back to the user, preceptors/mentors, and administrators.

Other embodiments of the present invention may be used to 1) quantify the diagnostic and procedural experience for each medical student, so that it becomes part of their permanent record (medical portfolio); 2) understand the breadth, depth, and variance of clinical experiences across all rotation specialties, rotation locales (clinics, hospitals, etc.), and students; 3) provide information to refine the clinical experiences and optimize the total training experience, especially to understand how well the curriculum objectives map to actual experiences; 4) expose students to the ICD code system, so that they are prepared to use it or similar systems as part of the Electronic Medical/Health Record (EMR/EHR) systems they will be using throughout their professional life; 5) capture and monitor in real-time the public health status of the communities in the vicinity of their training locales via accumulated statistical analysis of entries captured/sampled by the students, including trend and event trigger monitoring; and 6) capture the activities of specialized events, including off-shore medical mission trips and specialty fellows.

In other embodiments, the present invention provides a system that uses a common language/vocabulary/lexicon to associate clinical or training encounters by healthcare trainees and data that define/quantifies their progress, enhances their training and optimizes their training path. Common languages that may be used are WHO ICD codes, CPT codes, billing codes.

Other embodiments may include a collection of databases that are annotated with codes to connect patient encounters, learning materials, review materials, and performance metrics. Clinical encounters may be obtained from medical patient visits in hospitals or clinics, in transport vehicles, at retirement homes, and end of life care homes. Training encounters may include simulations, standardized patients, student-to-student exchanges, clinical skills classes, and events. Healthcare trainees may include medical students, nursing students, EMT students, fire, police, and emergency personnel. Data that defines/quantifies progress can be learning objectives, various logs (procedure, diagnostic, treatment), practice and real exams/scoring. Data that enhances training can be training materials presented to the trainee that is associated with their clinical or training encounter including matched educational material, review material, practice questions/answers/references, videos, and images. Data that optimizes training path may include evaluations of longitudinal (time-dependent) and/or measurables used to adjust or redirect training where it is most needed.

In other aspects, the present invention provides a system that uses a common language/vocabulary/lexicon to associate clinical or training encounters by healthcare trainees and data that define/quantifies the training and trainees which can then be used to adjust and optimize training and student success.

In yet aspects, the present invention provides a system that uses a common language/vocabulary/lexicon to associate clinical or training encounters by healthcare trainees.

In yet other aspects, the present invention provides a system that quantifies special or time dependence of diagnoses, procedures or drugs that are indicators of public health.

In yet other aspects, the present invention provides a system that has the ability to detect changes in dependences that indicate changes in public health; has the ability to provide summaries on public health.

In yet other aspects, the present invention provides a system that has the ability to set alerts that can inform on diagnoses, procedures or drugs which can be transmitted to people interested in public health (officials, researchers).

In yet other aspects, the present invention provides a system that includes computer or mobile device that captures and presents materials to/from trainees; a user interface; has the ability to select entries by searching hierarchical menus, recent entries, or most frequent entries by training module; has the ability to present entry associated materials for training, review, or evaluation/quantification of performance; includes one or more databases; has the ability to make annotations and associations; has training modules that include school class year, rotations, and lectures; has outputs that include searchable, filterable, sortable lists, graphs, maps; has outputs that can be delivered by download to excel or other databases or files; has outputs that include lists, graphs, or maps for school administrators; has outputs for researchers, and public health officials; includes an expanding time dependent set of databases of trainee observations and events; can be used by researcher to study training, public health, performance of drugs, procedures, diagnostics, and policy; and can be used for discovery of new biomedical associations and new drugs, procedures and diagnostics, school and government policy, and training.

In other aspects, the present invention provides a system implemented on a distributed computer network for capturing experiences of healthcare trainees. The system includes a plurality of electronic input devices having an interface for interacting with a user including an input screen having data input fields, selection fields and activation buttons and output screens. Also included are one or more back-end databases configured to store input data from the input devices and to provide output data to the input devices.

In other aspects, the present invention provides a system including a login system having user identification routines to establish user identity and user system access status. The system may also include a common language to identify and capture clinical or training encounters by healthcare trainees, said encounters included in the input data provided to the one or more back-end databases. The common language may be ICD codes, ICD code descriptions, CPT codes, CPT code descriptions or billing codes.

In other aspects of the present invention, the CD codes provide a common medical vocabulary that captures diagnoses, procedures, and drugs during patient encounters. The system may also be configured to use said captured codes to provide predetermined learning objectives, medical information or medical board review questions to a healthcare trainee.

In other aspects of the present invention, the system 1) is configured to provide real-time interactive information to a healthcare trainee associated with an inputted ICD code, 2) includes a collection of databases containing annotated ICD codes to connect patient encounters, learning materials, review materials, and performance metrics, 3) is configured to permit healthcare trainees to search ICD codes using hierarchical menus or to permit healthcare trainees to search ICD codes using keywords, 4) configured to permit healthcare trainees to search ICD codes by presenting frequently entered ICD codes, and 5) configured to permit healthcare trainees to link data entries to a selected ICD code.

In other aspects of the present invention, the system is configured to permit healthcare trainees, for each encountered patient, the ability to input: 1) the ICD code that describes their diagnosis and treatment procedure; 2) codes for WHO listed and/or FDA approved drugs; 3) the patient gender and age; 4) free-text notes; 5) the clinic/hospital and rotation type as entered by the user and whether the training environment is a hospital or ambulatory; 6) their precise location; and 7) date/time of entry.

In other aspects of the present invention, the system is configured to include a personally identifiable information detection system, said detection system prevents entry of personally identifiable information.

In other aspects of the present invention, the system the back-end database is configured to generate reports that enable the tracking and editing of data entries.

In other aspects of the present invention, the system is 1) configured to allow administrators to monitor the cumulative entries from all trainees; 2) configured to monitor trends and trigger alerts from defined thresholds; 3) configured to include displays adapted to monitor current activities being logged and to provide statistics that track and graph system-wide usage; 4) configured to continuously process and provide data on a plurality of displays; and 5) configured to provide a healthcare trainee an interactive summary page of one or more of the entries entered by the trainee, the one or more entries are editable, searchable, and have a variety of ranked column views.

In other embodiments of the present invention, entries are associated with predetermined learning objectives or entries have predetermined codes that qualify for fulfillment.

In other embodiments of the present invention, learning objectives or log entries are fulfilled by a manual entry by a trainee with a justification that is comprised of a reference, a reading, video or a lecture.

In other embodiments of the present invention, the system is 1) configured to provide preceptors, faculty or administrators the ability to evaluate a trainee via a check off and text entry table; 2) configured to provide preceptors, faculty and administrators access to trainee log entries, summaries and learning objective and log fulfillment tables to aid in the evaluation of a trainee; 3) configured to allow evaluations to be conducted via a secure one-time link to the evaluation form; 4) configured to automatically send emails to preceptors, faculty, and administrators with a link to initiate the evaluation process; 5) configured to send reminders to evaluators if they have not submitted their evaluation by a set time; 6) configured to allow an administrator to track an evaluator's completion of one or more evaluations; and 7) configured to capture evaluation information to determine if a trainee passes or fails a rotation.

In other embodiments of the present invention, individual clinical training sites or preceptor performance or uniformity can be evaluated by the quantitative and qualitative performance of their trainees via the trainee's entries.

In other embodiments of the present invention, the system is configured to compute the average and standard deviation of trainee entries to determine if one or more trainees, preceptors or sites are significantly above or below average.

In other embodiments of the present invention, review facts are presented to trainees for review following an entry of a given diagnostic, procedure or drug code.

In other embodiments of the present invention, one or more facts are annotated by one or more codes to trigger the presentation of the one or more facts to a trainee.

In other embodiments of the present invention, review questions and answers are presented to trainees for review following an entry of a given diagnostic, procedure or drug code.

In other embodiments of the present invention, one or more facts are annotated by one or more codes to trigger the presentation of the one or more facts to a trainee.

In other embodiments of the present invention, a response by a trainee to review questions is captured as either a right or wrong answer.

In other embodiments of the present invention, a wrong answer is represented at a later time.

In other embodiments of the present invention, associations between sites, preceptors, and students for each clinical rotation are imported, generated, or maintained.

In other embodiments of the present invention, the system is 1) configured to allow a trainee to record their research experiences by entering titles, abstracts, manuscripts, data or other information regarding research activities; 2) configured to allow faculty to enter their research interests and capabilities and desire to accept a student to do research so that students can search for and identify research mentors; 3) configured to allow a trainee to enter their research interests and capabilities for comparison with faculty research interests and capabilities to identify matches; 4) configured to include a personally identifiable information detection system, said detection system prevents entry of personally identifiable information by searching for first names, last names, and patterns that resemble phone numbers, social security numbers; and 5) configured to monitor trends and trigger alerts from defined thresholds, said alerts comprising email, text or call to a person or another system.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views Like numerals having different letter suffixes may represent different instances of substantially similar components. The drawings generally illustrate, by way of example, but not by way of limitation, a detailed description of certain embodiments discussed in the present document.

FIG. 1 illustrates a flowchart of the overall user process for an embodiment of the present invention.

FIG. 2 illustrates one user logging entry interface for an embodiment of the present invention.

FIG. 3A illustrates a system summary dashboard for an embodiment of the present invention.

FIG. 3B illustrates a user-editable summary table (with test data) system summary dashboard for an embodiment of the present invention.

FIG. 3C illustrates a student performance metrics summary table; metrics include counts of the number of entries as well as what fraction of the top 25 most encountered diagnoses, procedures, and drugs that this student has encountered, as a measure of the breadth of their clinical exposure, for an embodiment of the present invention.

FIGS. 4A and 4B illustrate a database schema for an embodiment of the present invention.

FIG. 5 illustrates a welcome screen for an embodiment of the present invention.

FIG. 6 illustrates an exemplary screen for updating a particular rotation and location for an embodiment of the present invention.

FIG. 7 illustrates an exemplary screen for entering patient demographics for an embodiment of the present invention.

FIG. 8 illustrates an exemplary screen for entering the primary diagnosis for an embodiment of the present invention.

FIG. 9 illustrates an exemplary screen for entering procedures for an embodiment of the present invention.

FIG. 10 illustrates an exemplary screen that couples the previously entered procedures and drugs with diagnosis entries to show the user's response to a patient's diagnosis for an embodiment of the present invention.

FIG. 11 illustrates an exemplary screen that allows the user to review and added information from a recent patient encounter or to start on a new patient encounter for an embodiment of the present invention.

FIG. 12 illustrates an exemplary screen for a dashboard page that allows a user to review and edit entries at any time for an embodiment of the present invention.

FIG. 13 illustrates an exemplary screen for editing an entry for an embodiment of the present invention.

FIG. 14 illustrates an exemplary welcome screen similar to the screen described in FIG. 5.

FIG. 15 illustrates an exemplary screen allowing for the navigation to all user functions from the system dashboard for an embodiment of the present invention.

FIG. 16 illustrates an exemplary screen of the system page that provides access to time graphs of entries in statistics, time graphs of students who are active, access to real-time student entries, summaries and map for an embodiment of the present invention.

FIG. 17 illustrates an exemplary student page for an embodiment of the present invention.

FIG. 18 illustrates an exemplary mentor page for an embodiment of the present invention.

FIG. 19 illustrates an exemplary administrator page for an embodiment of the present invention.

FIG. 20 illustrates an exemplary developer page for an embodiment of the present invention.

FIG. 21 shows an exemplary screen illustrating a time graph of students who are active for an embodiment of the present invention.

FIG. 22 shows an exemplary screen illustrating real-time student entries, summaries, map which may refresh every 10 seconds for an embodiment of the present invention.

FIG. 23 shows an exemplary screen illustrating filtering and mapping from a User Entry Page for an embodiment of the present invention.

FIG. 24 shows an exemplary screen illustrating rotation performance statistics for an embodiment of the present invention.

FIG. 25 shows an exemplary screen illustrating rotation statistics-raw entries for each location for an embodiment of the present invention.

FIG. 26 shows an exemplary screen illustrating location statistics-raw entries for each location for an embodiment of the present invention.

FIG. 27 shows an exemplary screen illustrating the utility of the present invention for issuing public health alerts for an embodiment of the present invention.

FIG. 28 shows an exemplary screen illustrating review facts and board questions presented to students triggered by a patient encounter for an embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

In one embodiment, the present invention provides a web-based computer and mobile application to track the progress of trainees, monitor the effectiveness of their training locations and be a means of sampling public health status. In one preferred embodiment, the present invention uses the logging of ICD Diagnostic, Procedure and Drug codes as one of the means of tracking the experience of medical students' clinical rotations.

In another preferred embodiment, the present invention provides a system and method that may be in the form of a web-based app in which medical trainees make entries via a simple and quick interface optimized for portable electronic devices such as mobile devices and personal computers. For each patient interaction, users enter ICD diagnostic, procedure, and drug codes via a hierarchical or search entry interface. Patient demographics (age range and gender, but no personal identifiers), and free-text notes may also be inputted. Users and administrators can review and edit input via a series of output interfaces. The user interface and one or more back-end databases may be provided such as by dual redundant failover Linux servers.

In a preferred implementation, students and other users were able to master the interface in a relatively short period of around ten minutes, and thereafter complete entries in less than one minute. In one working example, five hundred-forty 3rd-year students each averaged 100 entries in the first four-week clinical rotation. Data accumulated in various disparate clinical locations has demonstrated the public health surveillance utility of the application.

This data shows that PC and mobile apps can be used to collect medical trainee experience in real-time or near real-time, quickly, and efficiently. For example, in a preferred embodiment of the present invention, after collecting 75,596 entries, less than 2% of trainees needed assistance to become proficient. Medical school administrators are also using the various summaries to evaluate students and compare different rotation sites.

A primary goal of the present invention is to capture the day-to-day clinical experiences though ICD codes, by providing a user interface. The interface is configured to have operability that is quick, accurate, and efficient, so that students and other users will view this as a positive, convenient tool, as opposed to a time-consuming burden.

The data captured was a balance between what is needed to meet the goals of the system, and that which can be quickly and easily entered by users, so as not to over-burden them. Key areas were identified, such as a simple, straightforward human interface, optimized code entry methods and clear output summaries that enable individual performance monitoring. Another constraint was to create a system that did not require HIPAA compliance, so the design captured enough meaningful patient demographics to understand the relevant diagnoses and procedures logged, without needing Personally Identifiable Information, PII.

The design of the user interface and back-end database and the hardware capabilities, including speed, internet bandwidth, and reliability were prime considerations. For universal applicability across various PC types and mobile devices, a browser may be used. Also, the presentation and operation may be separately optimized for large (PC) and small (mobile device) screens. Another consideration was entry compatibility and speed across all display form factors. A preferred implementation requires Internet access. However, a native application may also be used to remove the need for continuous Internet access, something not always available in remote locations.

In another version, the logging system or app may be implemented through a browser page that quickly and easily captures trainee (or physician) experiences via user input of ICD codes has been developed, tested and is in use. The system syncs with a SQL back-end database, allowing for the accommodation of an effectively unlimited number of users. In one embodiment, there are over 1300 users.

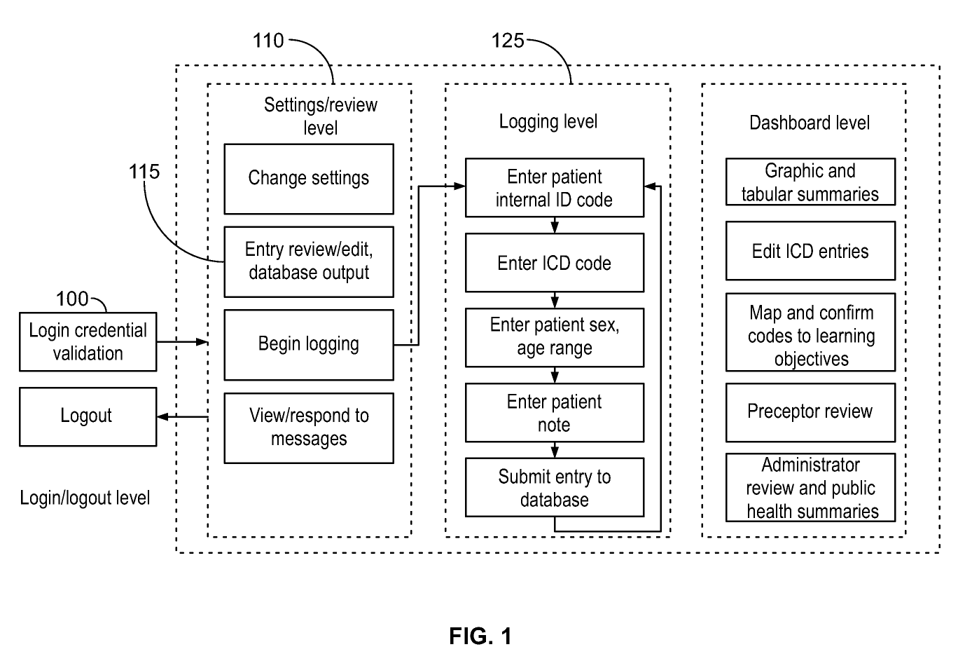

The user navigation flowchart is provided in FIG. 1. There are four primary ways in which users interact with the app after login: 1) they set their rotation type and location via the settings page; 2) they create new patient ICD log entries via the logging page; 3) the can measure their quantitative performance relative to all other medical trainees for each clinical rotation; and 4) they update and associate logged data with clinical learning experiences and build an example test bank of questions via the dashboard.

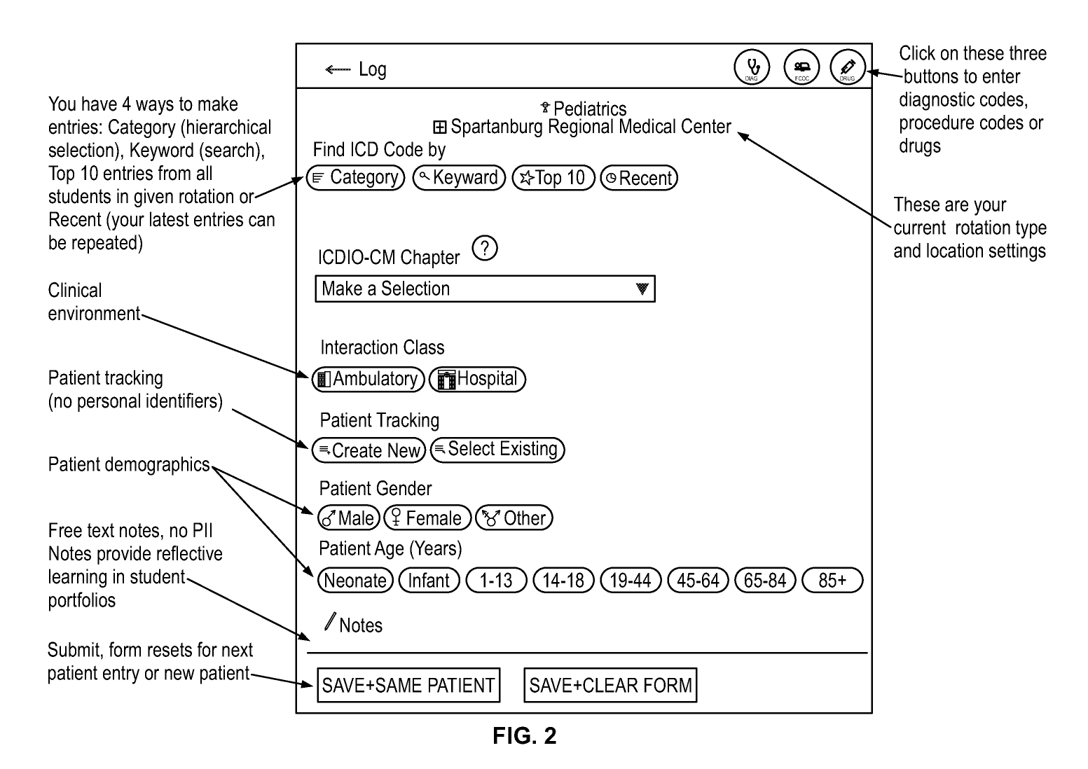

The primary web page used for logging provides the user with several methods for identifying the ICD code for a given entry—a hierarchical series of drop-down menus, a keyword-based search system; a list of the “Top 25” most frequent codes seen by all trainees in each rotation; and a recent entry selection. The system collects the following information from each user (student) as shown in FIG. 2. For each patient they experience; they select/enter the ICD code that describes their diagnosis and treatment procedure; and codes for WHO listed and/or FDA approved drugs; 2) the patient gender (M/F/O) and age (in relevant increments); 3) free-text notes; 4) the clinic/hospital and rotation type as entered by the user and whether the training environment is a hospital or ambulatory; 5) their precise location (as reported by the user's device); and 6) date/time of entry (populated using the server's time).

Users are able to select codes which would normally be considered non-billable to allow for deliberate ambiguity when a code of greater specificity is unfamiliar to the user or when there is insufficient clinical evidence for greater specificity. No personally identifiable information (PII) is collected on patients. A PII detection system prevents entry of PII into the free-text “Notes.” The system does not allow entries to contain text that includes patterns of numbers typical of phone numbers and social security numbers; or proper names. To do this, a database of 85,269 proper names was constructed from the analysis of the US Census; excluding certain common words that could also be proper names (certain stop words); and excluding eponymously-named diseases; e.g., Huntington's. The intent was not to create a new Electronic Health Records (EHR) system; which would require HIPAA compliance; but could limit acceptance of such a system in our many remote sites; if it was viewed as competing with existing systems.

The back-end database may be configured to generate reports that enable users to track and edit their entries; allows administrators to monitor the cumulative entries from all students; monitor trends; and trigger alerts from defined thresholds.

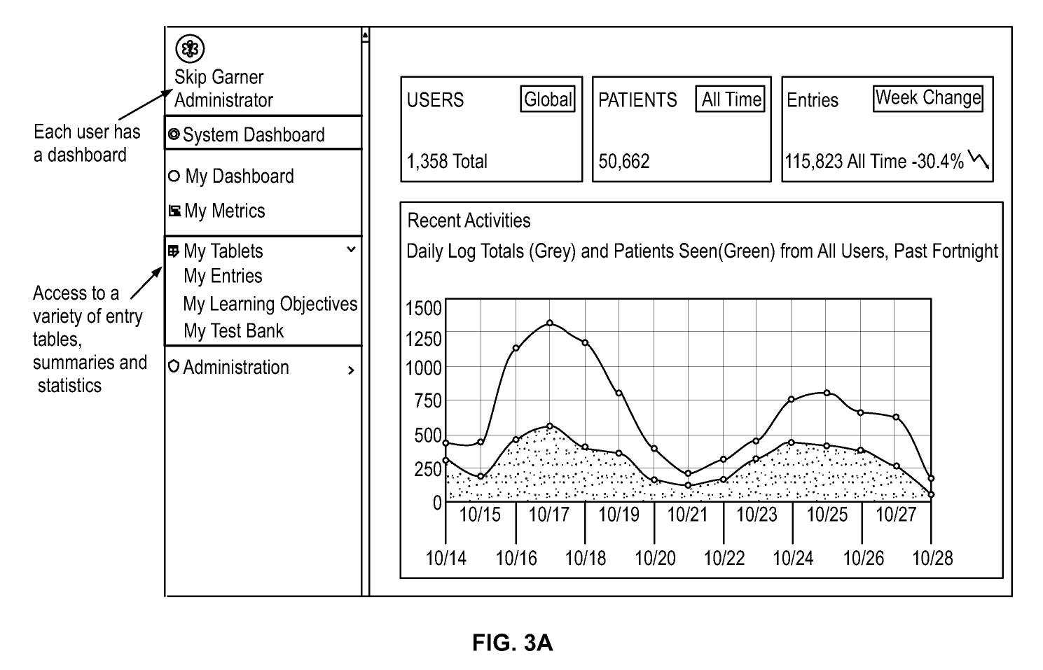

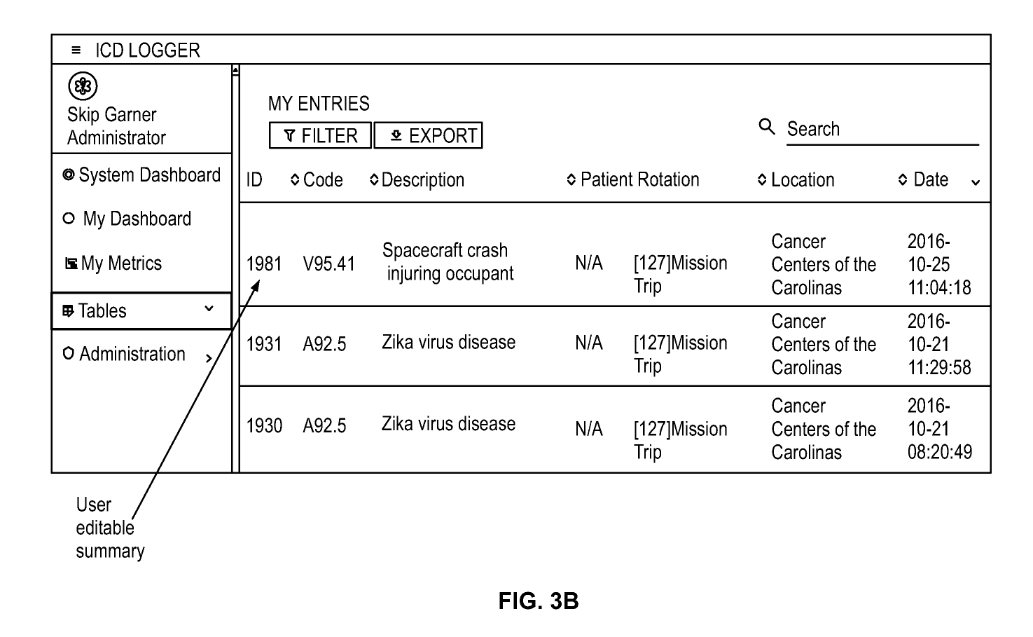

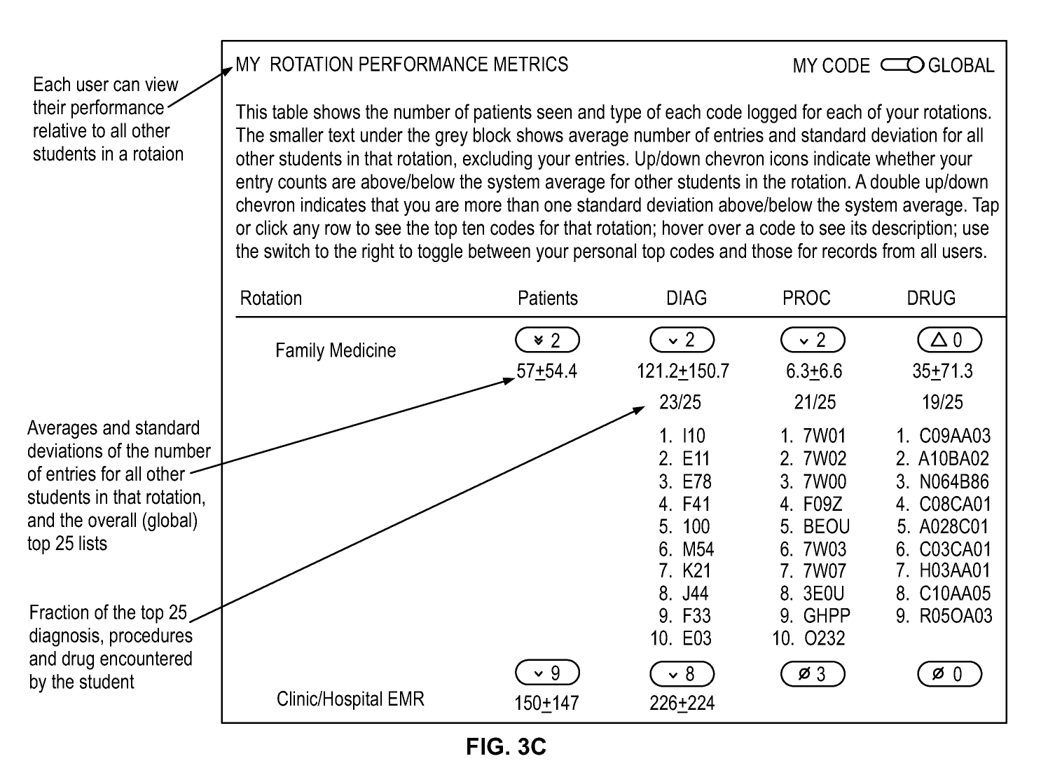

As shown in FIGS. 3A-3C, the system may include displays designed to monitor the current activities being logged (codes; date and time only) and statistics that track and graph system-wide usage which can be monitored by administrators or as public displays so that students and visitors at the medical school can get an instantaneous view of clinical activities.

The servers on which the system runs may be configured as two identical servers with fail-over capability, and other off-site backup and mirrored. Data interchange with the app's front-end interface is asynchronous to allow for rapid entry. The database, which may be MySQL, captures all the user logged information, and also contains all the user access information and other information required for the various displays.

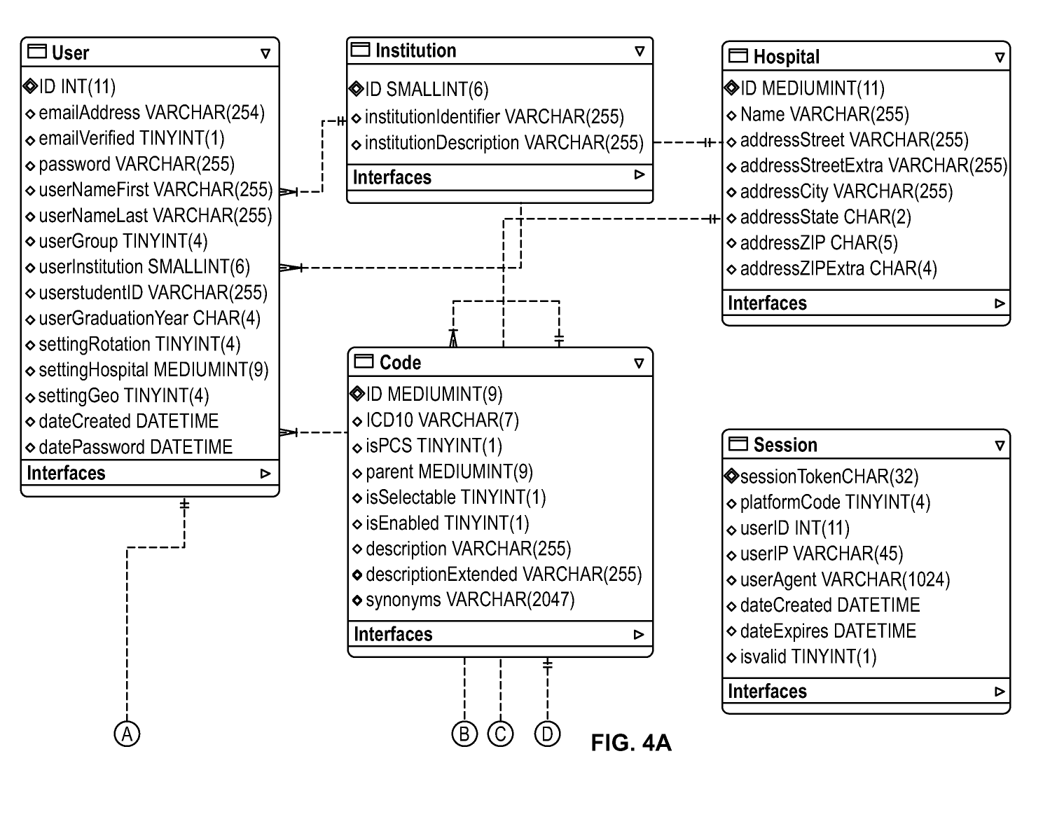

The database schema is given in FIG. 4. For one embodiment, the entire database (including entries and ICD codes) may be around 70.8 megabytes. Each new entry (including its index) on average is 0.25 kilobytes. The app uses approximately 1.2 megabytes on initial load, but with caching and mod_pagespeed optimization, subsequent loads are only 34 kilobytes. Thus, the system (and associated bandwidth to/from servers) was designed for hundreds of simultaneous queries by users, for greater than 50,000 users, following stress tests conducted using Loader.

Content that may be used with an embodiment of the system is given in Table 1, including both diagnostic and procedural codes, retrieved from the WHO website, and drugs downloaded from the WHO ATC website, and FDA approved drugs web site.

TABLE 1
Content of the data tables.

| | |
|---|---|
| ICD-10 Diagnostic Codes | 44,221 |
| ICD-10 Procedure Codes | 78,705 |
| WHO/FDA Approved Drugs | 4823 |
| Student Rotation Sites | >700 |
| Student Rotation Types | >80 |
| Learning Objectives | >500 |
| Student Users | >1200 |
| Faculty/Admin Users | >100 |

All codes are stored in the local database for maintaining referential integrity and providing detail-rich reporting functionality. For example, at the VCOM medical school, there are over 80 recognized rotation and activity types; these activities are conducted at over 700 different locations (clinics, hospitals, etc.). There are program-specific tables which house rotation types and specific clinical locations, as well as administrative tables to maintain user entities, granular access rights, forgotten password recovery codes, etc.

A keyword search procedure may be implemented through the use of the MySQL engine's native full-text index. The ICD descriptions were supplemented with synonyms to ease and speed finding the most specific code and enable common medical and non-medical terms to be used.

In another embodiment, data may be continuously processed and provided on a variety of displays (web pages) which may be in the form of one or more dashboards. Each student or users is provided with an interactive summary page of all their entries, which is editable, searchable, and has a variety of ranked column views. Students or users can also monitor their performance relative to all other students in the same rotation. Performance reports contain the number of entries made by the student or users and the fraction of the top 25 diagnoses, procedures and drugs which they have encountered as a measure of the breadth of clinical experience relative to all other students or users in a given rotation. There are also display pages for overall system use, recent entries, and other information. The full set of user entries can be exported to an Excel spreadsheet so that a variety of custom analyses can be performed by system administrators or faculty who wish to study the activities of the students during their various activities.

In actual use, it has been found that it initially takes approximately ten minutes to master and begin logging. Thereafter the time required to make new log entries drops to <1 min/entry and users primarily select ICD codes using the search feature. Diagnostic entry selection is easier than procedure entries, especially as more specificity is required. Students interact with the primary log entry page over 95% of the time, and only rarely review and edit/update entries via the dashboard interface.

In one use that was studied, a team of eight 3rd year medical students was engaged to log their experiences and provide feedback. Students have completed two clinical rotations (family medicine, internal medicine, psychiatry, geriatrics, pediatrics, etc.) of one-month duration each at 13 different hospital/clinic sites. They have entered 2623 diagnostic, procedure and drug codes in the 40 days they have been in the clinic. The student entry rate varied significantly, between 97 and 916 entries, and it did not correlate with any particular type of rotation, but appears to reflect the variation in rigor among students. There were a total of 586 drug codes entered, with the most frequent being Amoxicillin, Cefdinir, Ondansetron and various forms of Acetaminophen. The most frequent diagnostic codes were (I10) essential primary hypertension, (E78x) various forms of hyperlipidemia, (E11x) various forms of Type 2 Diabetes, and (Bx) various viral infections. This sampling of diagnoses reflects the known obesity prevalence in the area monitored.

An additional system utility demonstration was obtained from 2nd-year students that participated in a medical outreach trips to El Salvador, Honduras, and the Dominican Republic, where VCOM maintains a permanent local presence at its clinics. There have been ten mission trips, 7211 codes were entered by 239 student users.

This has also provided insight into the Public Health potential for the present invention which has enabled fundamental observations: Females come to the clinics at twice the rate of males; the code (I10) essential primary hypertension was the most frequent, as expected given the worldwide obesity crisis. Given the socioeconomic level of the indigenous population that visit the clinics, it was not surprising to see that the next most frequent entries included were (J069) acute upper respiratory infection, (B89) unspecified parasitic disease, and (K21) gastroesophageal reflux disease with esophagitis, for these are typically seen in first-world populations. There were a number of unique codes logged, including (A30) Leprosy, and a number of genetic or complex disorders: (Q90) Down's syndrome, (C5061) malignant breast neoplasm, and (Q66) congenital deformities of the feet, to name a few

Students and user have accepted the app as their primary method for capturing their clinical experiences and building their experience portfolio. The app, therefore also takes on a role in the formal documentation for the medical school. Preceptors may use the ICD Logger of the present invention in reviewing student performance during a given rotation, and also use this information as an opportunity to engage students in reflective learning based on cases they have logged. Administrators can also evaluate the uniformity of the clinical experiences across various training sites using this data, both quantitatively (number of entries) and the considering the spectrum of different clinical cases encountered (using the top 10 list).

The embodiments of the present invention may be used in many ways, including use in other medical training areas such as Emergency Medical Technicians, nursing, and dentistry. For these other allied health professions require clinical experiences, which could be captured in by using codes such as ICD-10 codes. The embodiments of the present invention may also be used in other medical/non-medical areas such as billing/coding training, survey taking, etc.

The embodiments of the present invention may also be used as a public health monitoring/surveillance tool, for the data emerging is consistent with the general population health in the areas surveyed.

In yet another use, data collection was completed every week, and a designated doctor manually uploaded the patients seen during the week from the HealthMaster database, a locally developed and established EMR system, and to the system of the present invention. The similarities and differences between two underserved sites are shown in Table 2.

TABLE 2
The Top 10 Diagnoses for the Ghana and Appalachia sites. Using the “Top 10” feature in the Logger page,
these lists were composed to demonstrate the similarities and differences between the locations

| Clinic Rotation at HMC | Family Medicine in Appalachia |
|---|---|
| Top 10 Diagnoses | Top 10 Diagnoses |
| Plasmodium falciparum malaria | Encounter for general examination without complaint, suspected or |
| Essential (primary) hypertension | reported diagnosis |
| Other disorders of urinary system | Essential (primary) hypertension |
| Acute upper respiratory infections of multiple and unspecified sites | Type 2 diabetes mellitus |
| Other anemias | Disorders of lipoprotein metabolism and other lipidemias |
| Disorders of lipoprotein metabolism and other lipidemias | Biochemical lesions, not elsewhere classified |
| Vasomotor and allergic rhinitis | Dorsalgia |
| Other and unspecified soft tissue disorders, not elsewhere classified | Other joint disorder, not elsewhere classified |
| Unspecified acute lower respiratory infection | Persons encountering health services in other circumstances |
| Open would of head | Encounter for screening of malignant neoplasms |
| | Long term (current) drug therapy |
| Top 10 Prescriptions | Top 10 Prescriptions |
| Ibuprofen | Lisinopril |
| Ciprofloxacin | Levothryoxine sodium |
| Cetirizine | Amoxicillin |
| Folic acid | Gabapentin |
| Metronidazole | Dexamethasone |
| Amoxicillin | Atorvastatin |
| Diazepam | Hydrochlorothiazide |
| Bendroflumethiazide | Azithromycin |
| Naproxen | Prednisone |
| Cefuroxime | |

Tema, Ghana is considered a suburban locality, with the most populous area being Community 1. Appalachia was chosen because it is a socioeconomically disadvantaged population in the US; in some respects, it has similarities to a third-world country. Both sites have essential (primary) hypertension as their second most frequent diagnosis as well as disorders of lipoprotein metabolism and other lipidemias, which can be attributed to the increasing rate of obesity and lack of exercise. Similar drugs used to treat these conditions are frequently administered at both sites, such as Bendroflumethiazide, Hydrochlorothiazide, Atorvastin, and Lisinopril.

They both use various antibiotics, such as Amoxicillin, Ciprofloxacin, and Azithromycin. However, the use of these drugs reflects the differences between diagnoses at the two sites. Although it is advanced, HMC Ghana has limited resources and cannot always obtain the best medications, limiting what prescriptions are available to the patients. Some of the diagnoses made in Ghana are respiratory in nature, possibly due to the working conditions, excessive dust in the environment and the plurality of viral infections in tropical environments. The prescriptions made to the patients in Appalachia are more orthopedic; for example, the diagnoses of dorsalgia and other joint disorders. Regular visitation of the doctor's office or “check-ups” (encounter for general examination without complaint) is not common in Ghana, due to the cultural standard of if one is not visibly ill, there is no need to go to the doctor.

Most developing countries lack the technical expertise, funding and technological infrastructure needed for implementing EMR systems. However, some countries have proven that is possible to have a locally developed EMR, such as the one in Ghana. This EMR functions very well for the size of the clinic, although it does have technical issues with certain features. Using the system and methods of the present invention supports an established system by providing back-up on a server that is less likely to experience technical issues. It also provides a universal medical language (ICD codes) that enable world-wide comparisons. Due to its transparent and intuitive design and minimal cost, the implementation of the present invention would be beneficial for all concerned. The present invention may be used to provide public health data at a per-patient resolution for future research for a predetermined location such as the Sub-Saharan, and a facility or institution would have a secure back-up of their patient information centrally located outside of the country. Transcribing data from one EMR system to another is an arduous and prolonged task. However, capturing universal information (ICD codes) for a patient and having a separate backup of patient concluded that bi-weekly data conversion and collection was information was considered valuable. After initial tests, we the best option.

The embodiments of the present invention may be used as an ancillary EMR system as was shown by testing in a new hospital in Ghana. This allowed the hospital to have an off-site backup of critical medical records. Capturing the information in a universal way, using WHO's ICD-10 codes, enables the present invention to compare the public health picture in a predetermined location such as Ghana relative to a US population.

The embodiments of the present invention may also be used as a stand-alone EMR system, sufficient for developing nations, provided that it is accompanied with a database that relates the internal CREDO ICD Logger patient ID with their true ID.

By keeping the conversion database local, confidential and encrypted, it is possible to separate the bulk of the patient information via ICD-10 codes from the confidential information. This, in turn, demonstrates that a large portion of the information, information of particular value to continuously monitoring and surveying public health down to individual anonymous patients is possible.

Login/logout level pseudo-code for use at step 100 of FIG. 1 may include:
Users login with a username and password
After login, users are in settings level
After a user is finished logging or viewing reports, they can return to settings level and then log off

Settings level 110, as shown in FIG. 1, pseudocode may include:
Administrators can set alerts, request database download
Settings allow a user to change a password, location (including a selection from a pre-determined set of GPS coordinates obtained from a mobile device), rotation; settings are saved, exit settings and return to settings/logoff level
User can view about and help
A user can log off
User can view and respond to messages
User can view various database reports (output)
User can go to entry-level Enter settings level
User can go to settings, messages, about, help, reports, logoff

Database reports 115, as shown in FIG. 1, pseudocode may include:
User can enter reports level and request several types of view summaries
User entries over time, by code, by location, by sex, by age group
Administrators can enter reports level and request several types of view summaries
Administrators can request entries by student, location, code, time, location, sex, age; and various sets of these
Administrators can view alerts

Backend database and updater pseudocode and schema Backend database contents may include:
Table of users, passwords, and privileges
Table of locations (hospitals, clinics, remote places, GPS coordinates)
Table of ICD codes (number, hierarchy, description)
Table of entries (entry ID, ICD code entered, M/F/O, age, location, user, time/date stamp, rotation name)
Database logic
Consistency checking
Alert detection
Updater (administrative control)
Table loader of new/updates set of ICD codes, new/change/remove users, set of locations, set of rotation names)
Ability to edit, change, add, delete codes, users, locations, rotation names
Downloader and backup (administrative control)
Export databases to excel
Backup and sync database across servers

Logging level 125, as shown in FIG. 1, pseudocode may include:
Logging level entered
User can enter with pulldown menus, buttons and text entry boxes in any order ICD code, sex, age, notes; and optionally not sex, age, notes
Entry of ICD code can use search for a code
Entry of ICD code can use autocomplete
Entry of ICD code can use recent entries
User submits with button, which makes entry into a database, and resets logging page for another entry, i.e., 2 and 3 loops until exit this routine
Exit routine back to settings level

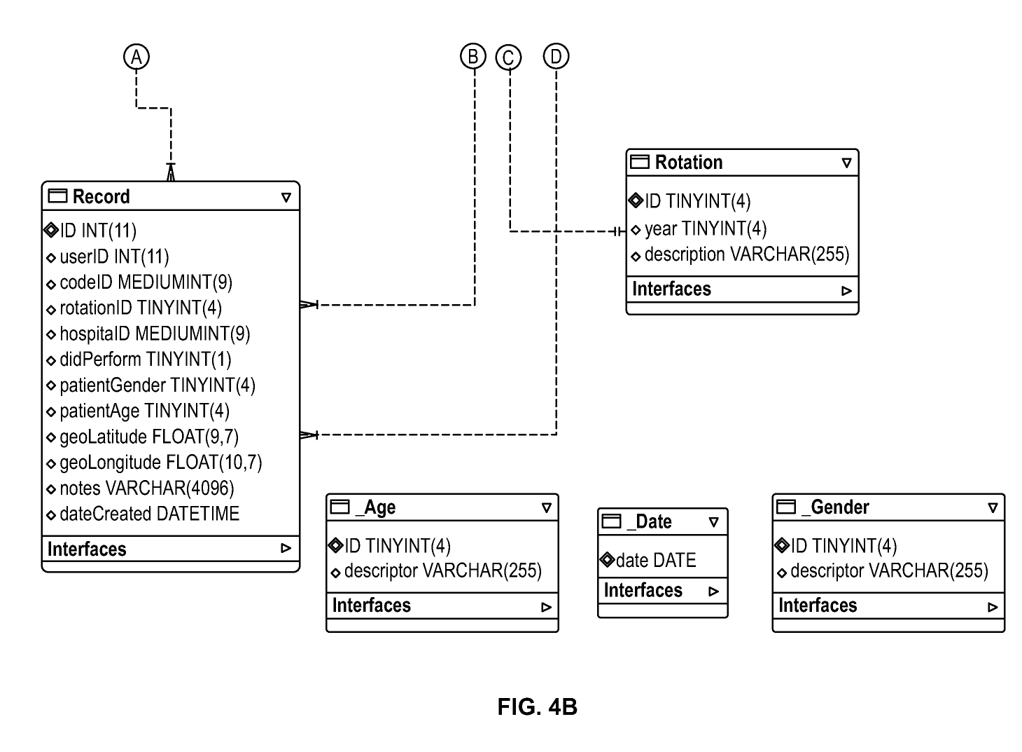

FIGS. 5-28 illustrate exemplary web pages or screens that may be used with various embodiments of the present invention. FIG. 5 shows a welcome screen. It provides areas, sections, or buttons where a user may change rotation and location 500, a section where help and review instructions may be obtained 502, dashboard access 504 where the user can review and edit patient encounters, patient encounter access 506 where a user can enter patient-specific information discussed below, and the screen also provides the opportunity to confirm the rotation and location 508.

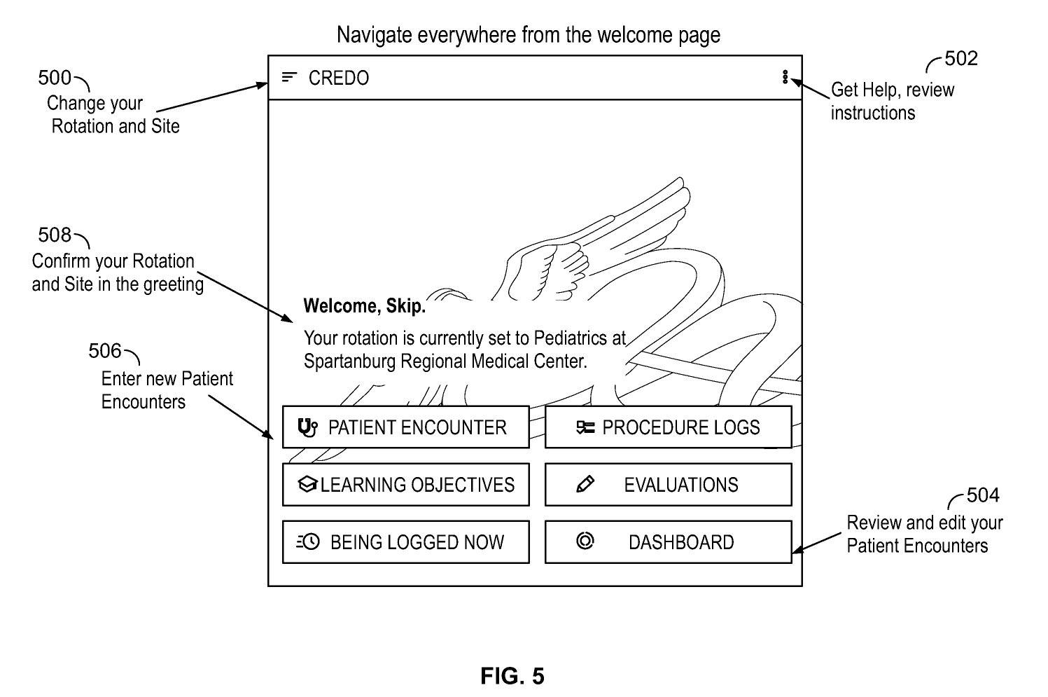

FIG. 6 illustrates an exemplary screen for updating a particular rotation and location. To update the rotation and location, the account setting screen is used where a user is able to select a particular rotation type and location of the rotation 600.

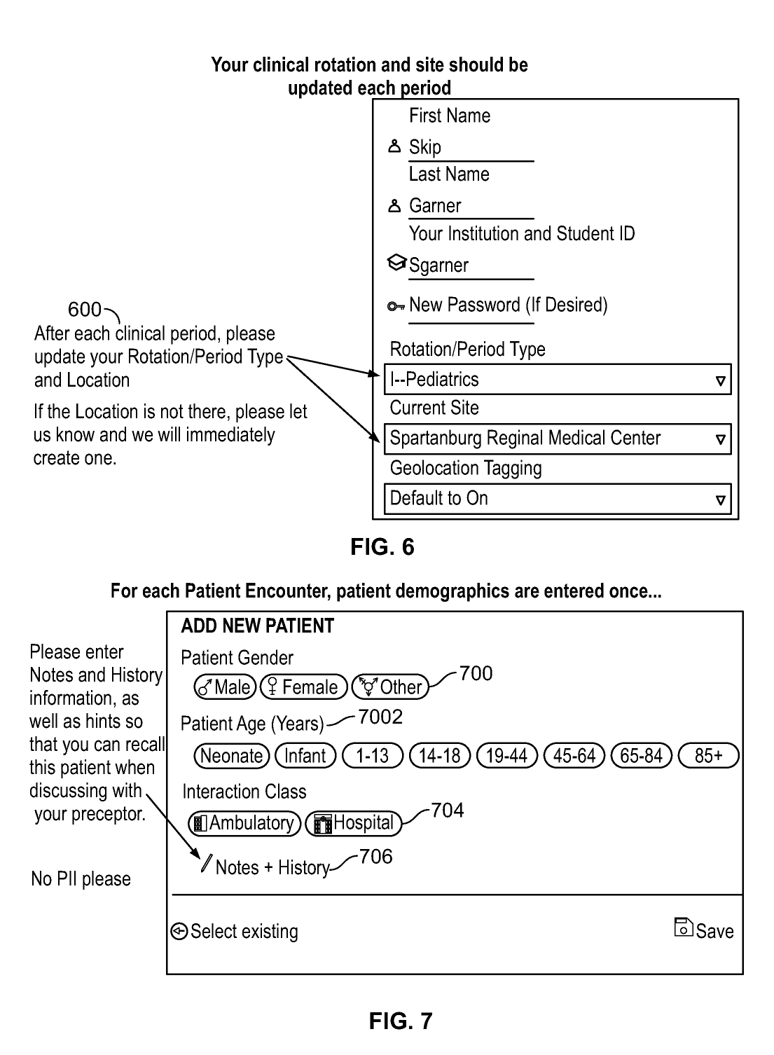

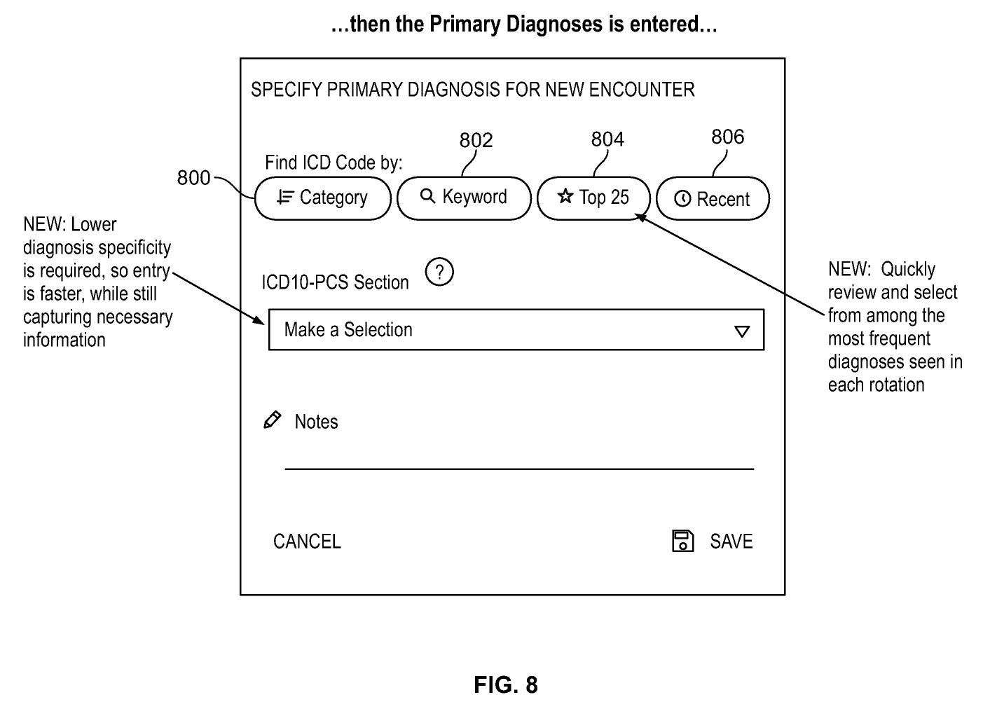

FIGS. 7-11 illustrate exemplary screens for entering patient encounter information where a user is led through a series of steps where diagnoses, procedures, and drugs for each patient are entered. FIG. 7 illustrates a screening for entering patient demographics such as gender 700, age 702, interaction class 704 and a section for adding notes and history 706. FIG. 8 illustrates entering the primary diagnosis. As shown, ICD codes may be determined by category 800, keywords 802, top 25 or most frequent diagnosis seen in the rotation 804, and most recent 806.

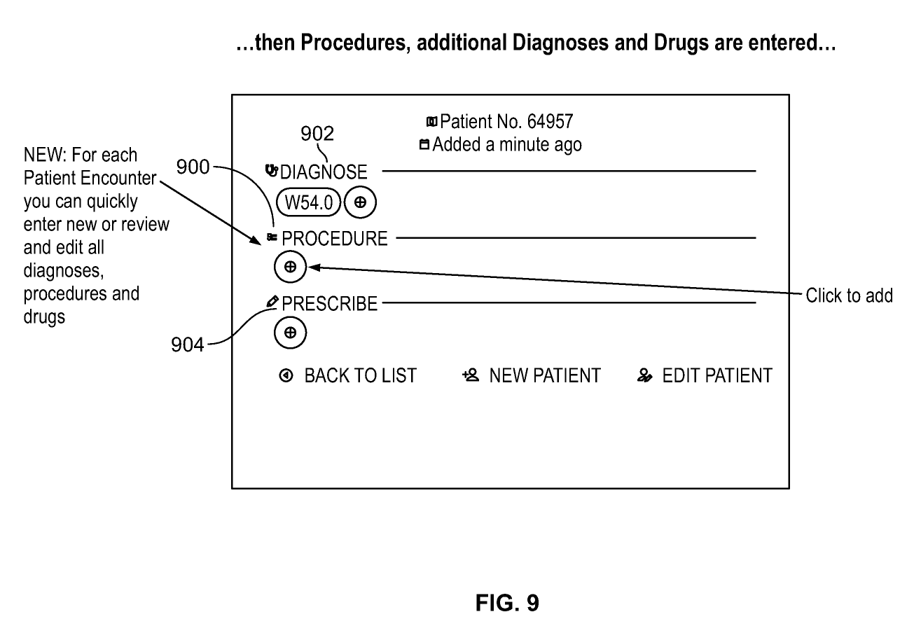

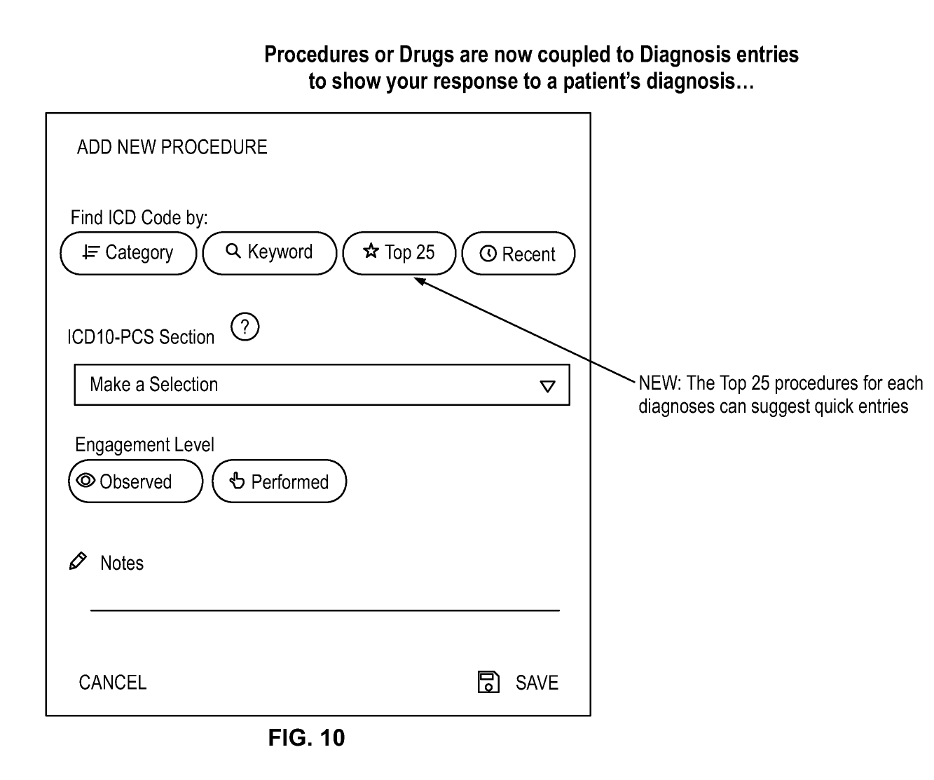

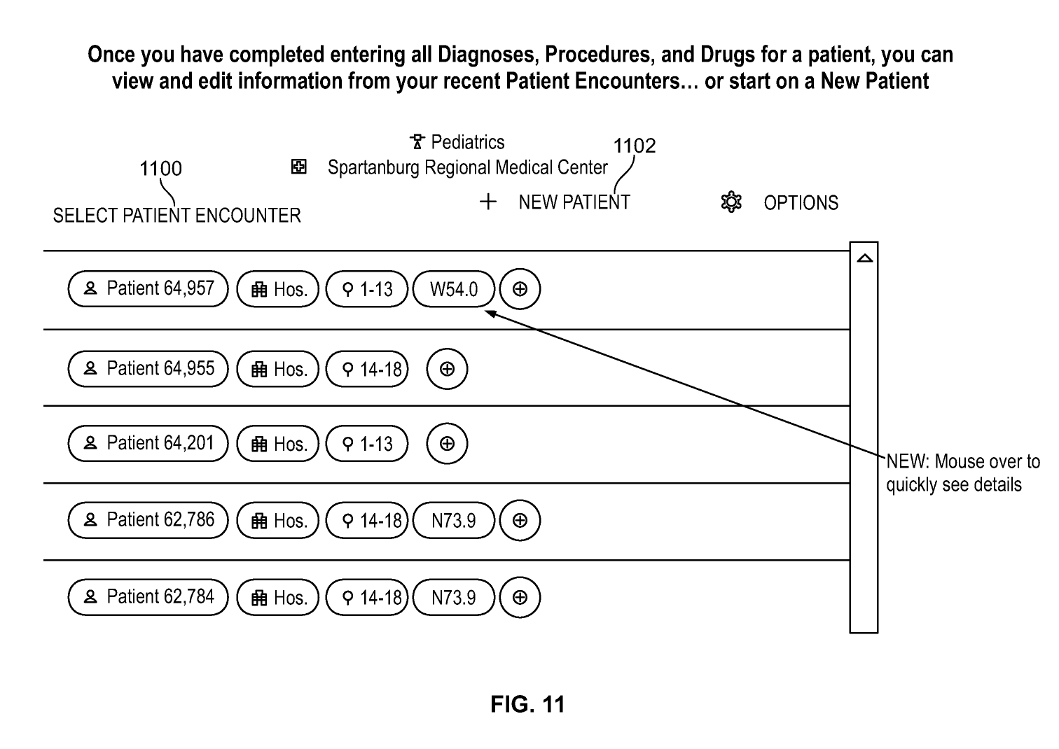

FIG. 9 illustrates an exemplary screen for entering procedures 900, one or more additional diagnoses 902 and drugs 904. FIG. 10 illustrates an exemplary screen that couples the previously entered procedures and drugs with diagnosis entries to show the user's response to a patient's diagnosis. FIG. 11 illustrates an exemplary screen that allows the user to review and added information from recent patient encounter 1100 or to start on a new patient encounter 1102.

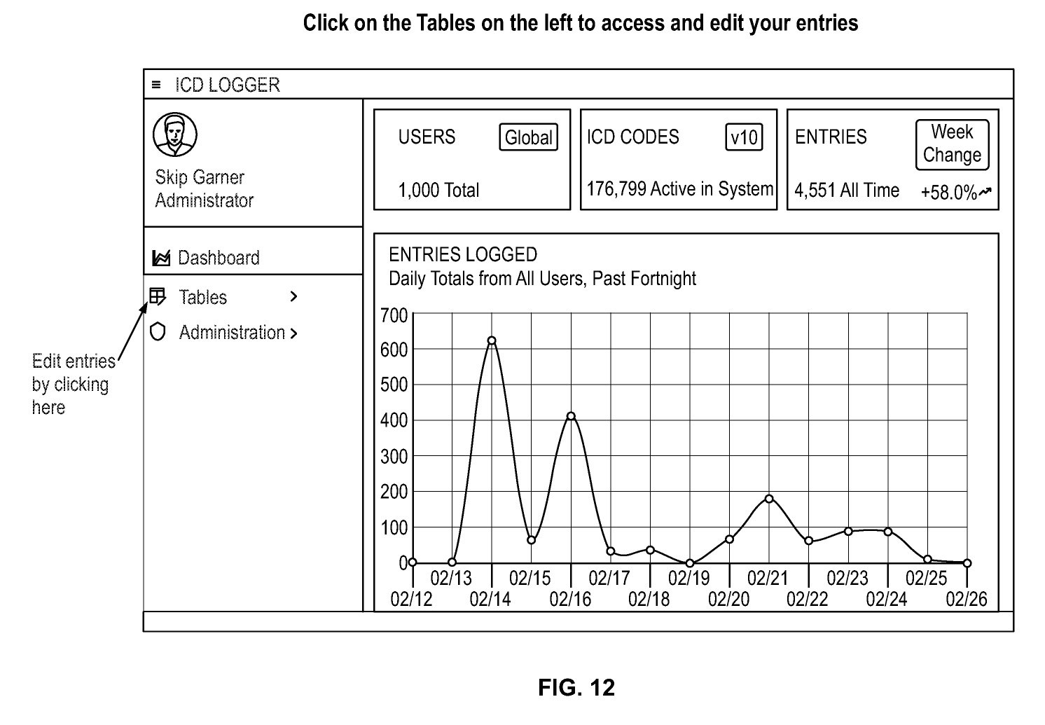

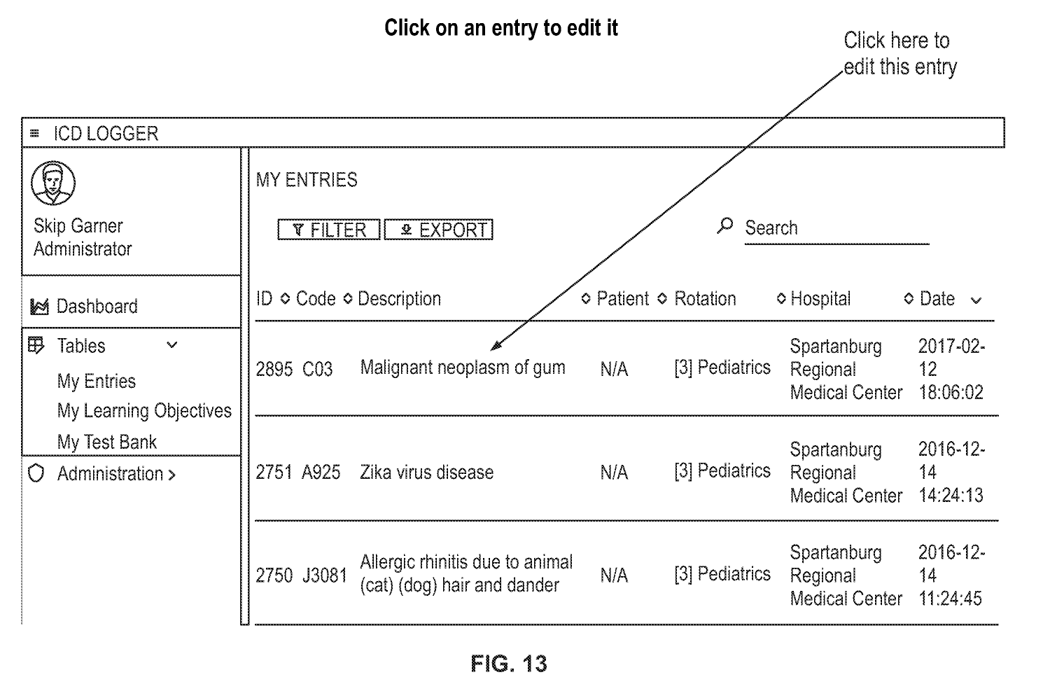

FIG. 12 illustrates an exemplary screen for a dashboard page that allows a user to review and edit entries at any time. Information that may be provided on the dashboard includes the ability to edit entries and review tables and charts providing global and individual information. FIG. 13 is an exemplary screen for editing an entry.

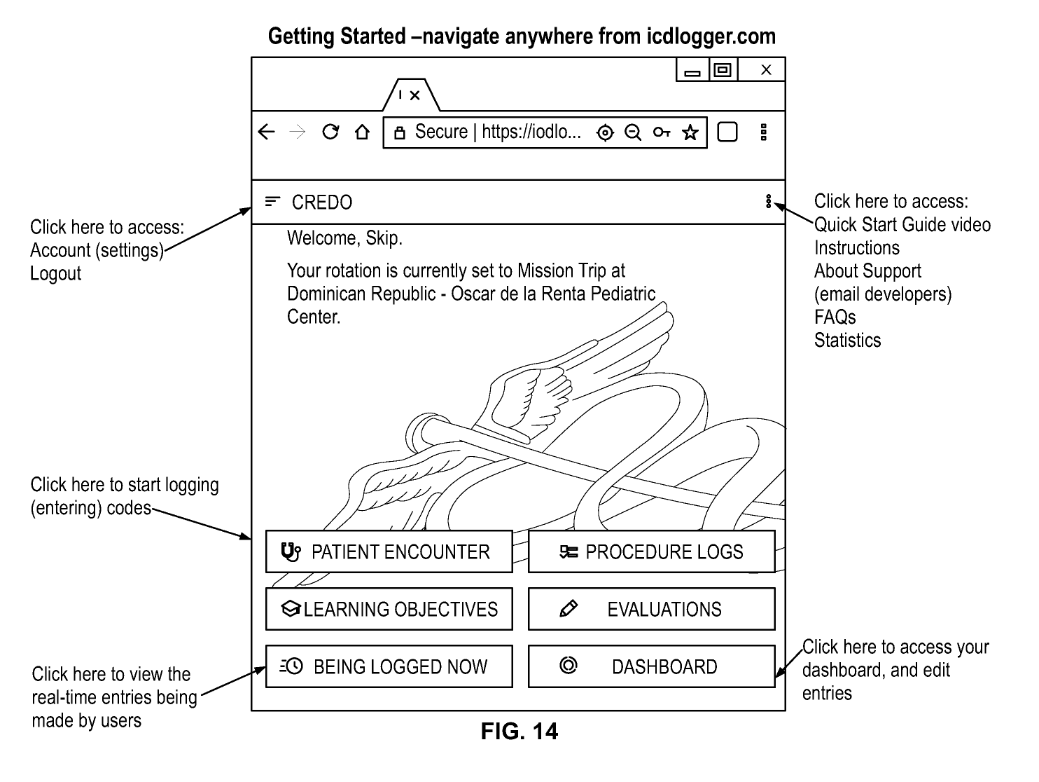

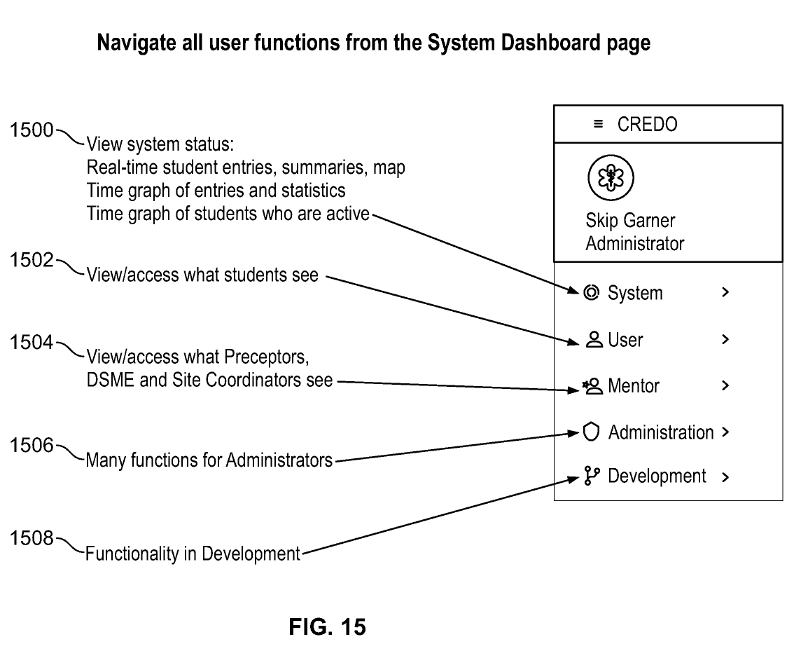

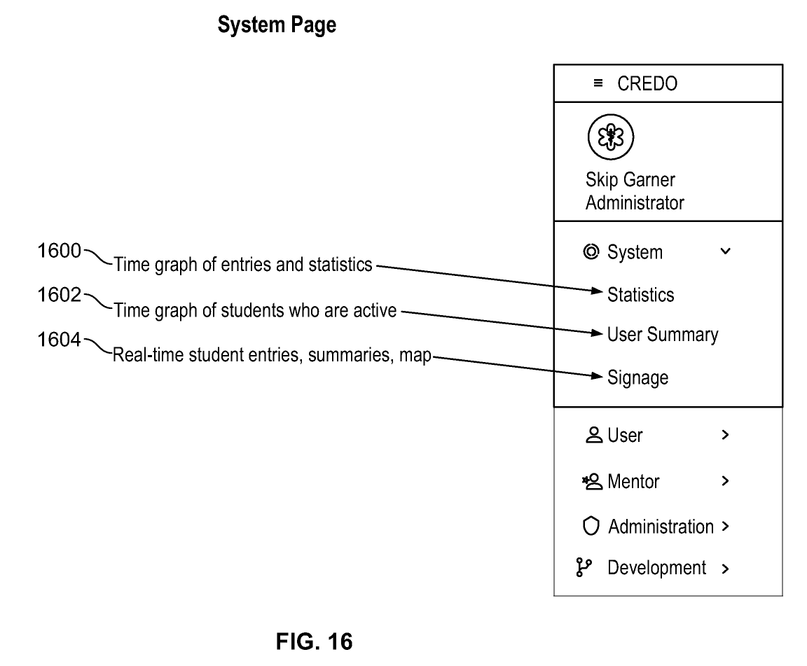

While FIGS. 5-13 are primarily directed towards student users, FIGS. 14-28 are primarily directed to administrative functions but may be used by student users as well. FIG. 14 is an exemplary welcome screen similar to the screen described in FIG. 5. FIG. 15 illustrates an exemplary screen allowing for the navigation to all user functions from the system dashboard. System access 1500 allows access to real-time student entries, summaries, map, time graphs of entries and statistics, and time graphs of students who are active. User button 1502 allows for reviewing and access to see what a particular student sees. Button 1504 allows a user to review and access what preceptors, DSME and site coordinators see. Button 1506 provides for access to administrative functions, and button 1508 is reserved for functionality development. FIG. 16 is an exemplary screen of the system page that provides access to time graphs of entries in statistics 1600, time graphs of students who are active 1602, and access to real-time student entries, summaries and map 1604.

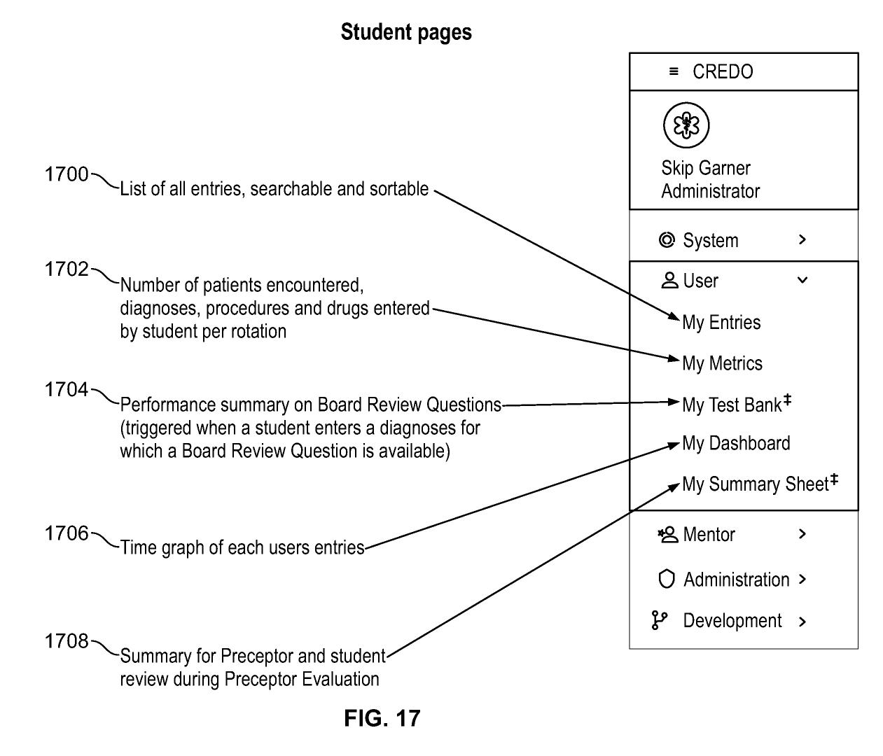

FIG. 17 illustrates an exemplary student page. Buttons that may be provided include 1700 List of all entries, searchable and sortable; 1702 Number of patients encountered, diagnoses, procedures and drugs entered by a student per rotation; 1704 Performance summary on Board Review Questions (triggered when a student enters diagnoses for which a Board Review Question is available); 1706 Time graph of each entry; 1708 Summary for Preceptor and student review during Preceptor Evaluation.

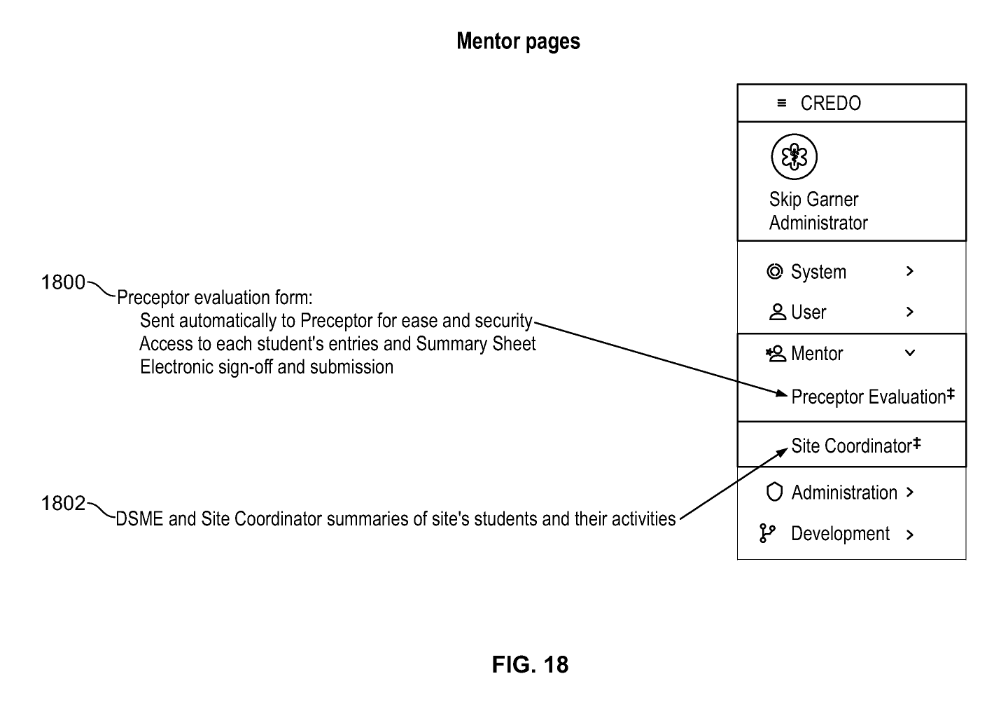

FIG. 18 illustrates an exemplary mentor page. Buttons that may be provided include: 1800 Preceptor evaluation form, sent automatically to Preceptor for ease and security, access to each student's entries and summary sheet and electronic sign-off and submission; and 1802 DSME and Site Coordinator summaries of site's students and their activities.

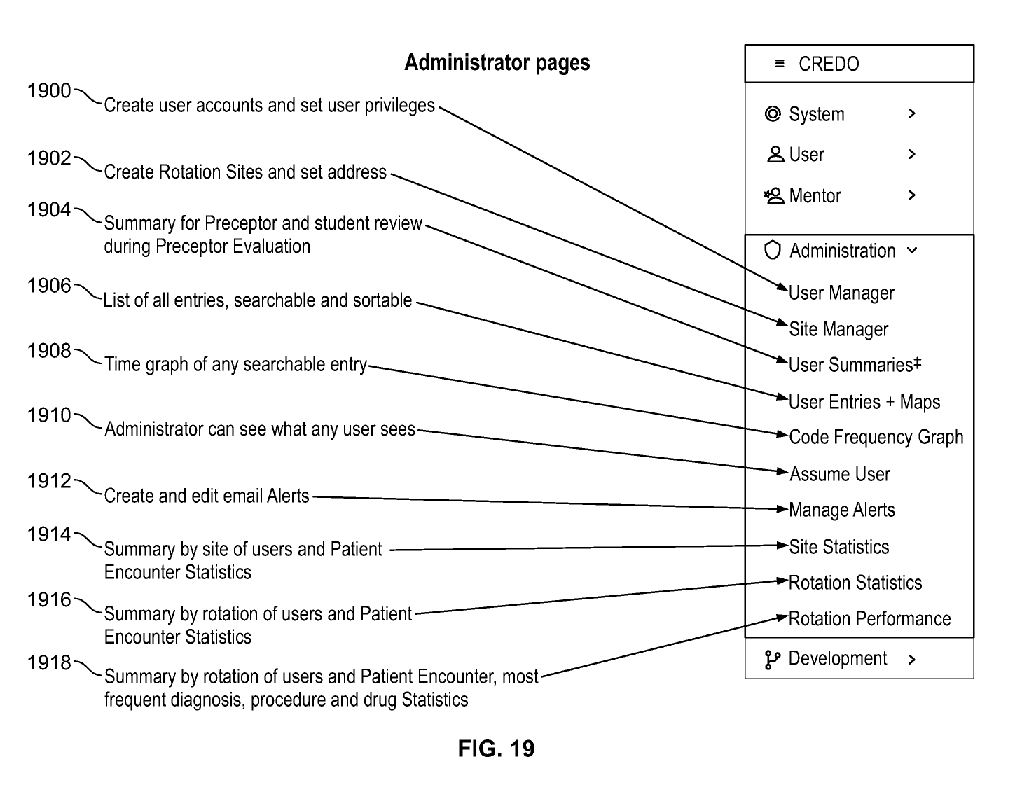

FIG. 19 illustrates an exemplary administrator page. Buttons that may be provided include: 1900 Create user accounts and set user privileges; 1902 Create Rotation Sites and set address; 1904 Summary for Preceptor and student review during Preceptor Evaluation; 1906 List of all entries, searchable and sortable; 1980 Time graph of any searchable entry; 1910 Administrator can see what any user sees; 1912 Create and edit email Alerts; 1914 Summary by site of users and Patient Encounter Statistics; 1916 Summary by rotation of users and Patient Encounter Statistics; and 1920 Summary by rotation of users and Patient Encounter, most frequent diagnosis, procedure and drug Statistics.

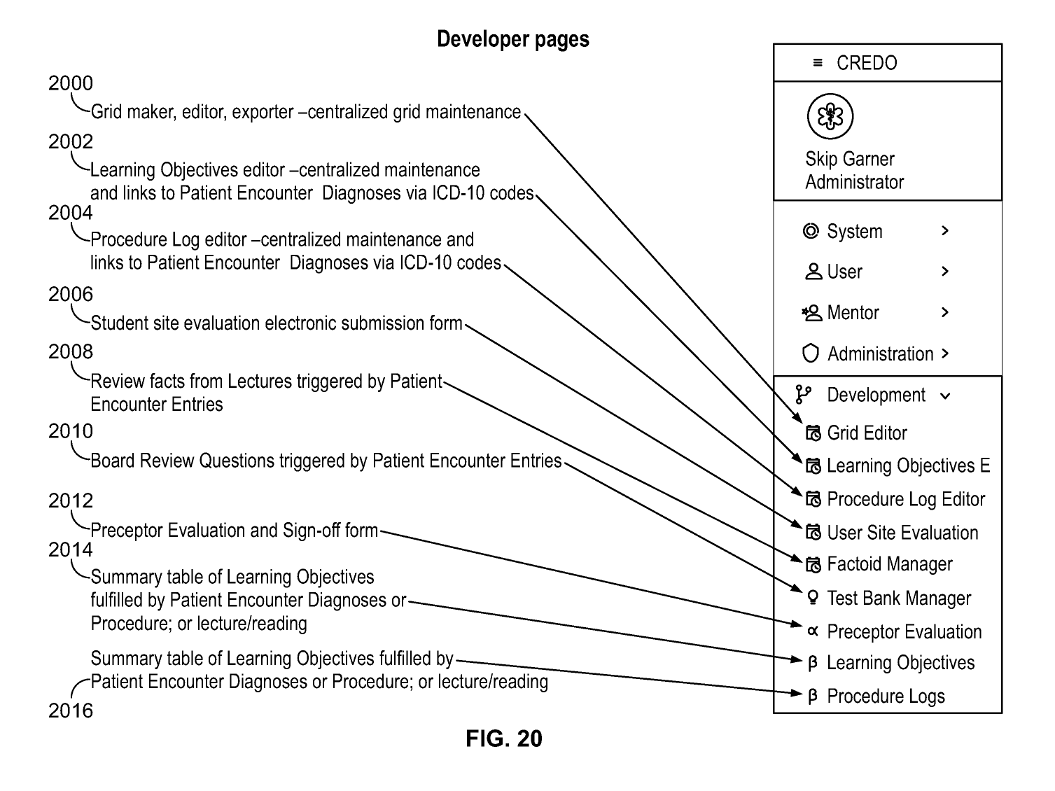

FIG. 20 illustrates an exemplary developer page. Buttons that may be provided include: 2000 Grid maker, editor, exporter—centralized grid maintenance; 2002 Learning Objectives editor—centralized maintenance and links to Patient Encounter Diagnoses via ICD codes; 2004 Procedure Log editor—centralized maintenance and links to Patient Encounter Diagnoses via ICD codes; 2006 Student site evaluation electronic submission form; 2008 Review facts from Lectures triggered by PatientEncounter Entries; 2010 Board Review Questions triggered by Patient Encounter Entries; 2012 Preceptor Evaluation and Sign-off form; 2014 Summary table of Learning Objectives fulfilled by Patient Encounter Diagnoses or Procedure; or lecture/reading; and 2016 Summary table of Learning Objectives fulfilled by Patient Encounter Diagnoses or Procedure; or lecture/reading.

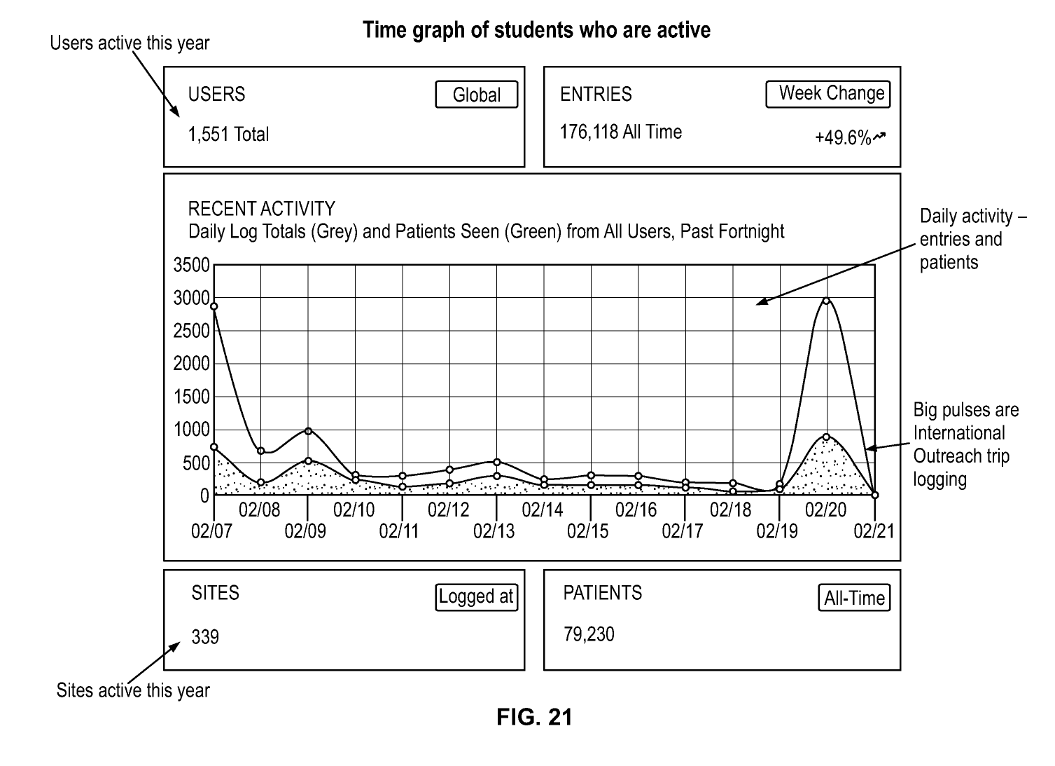

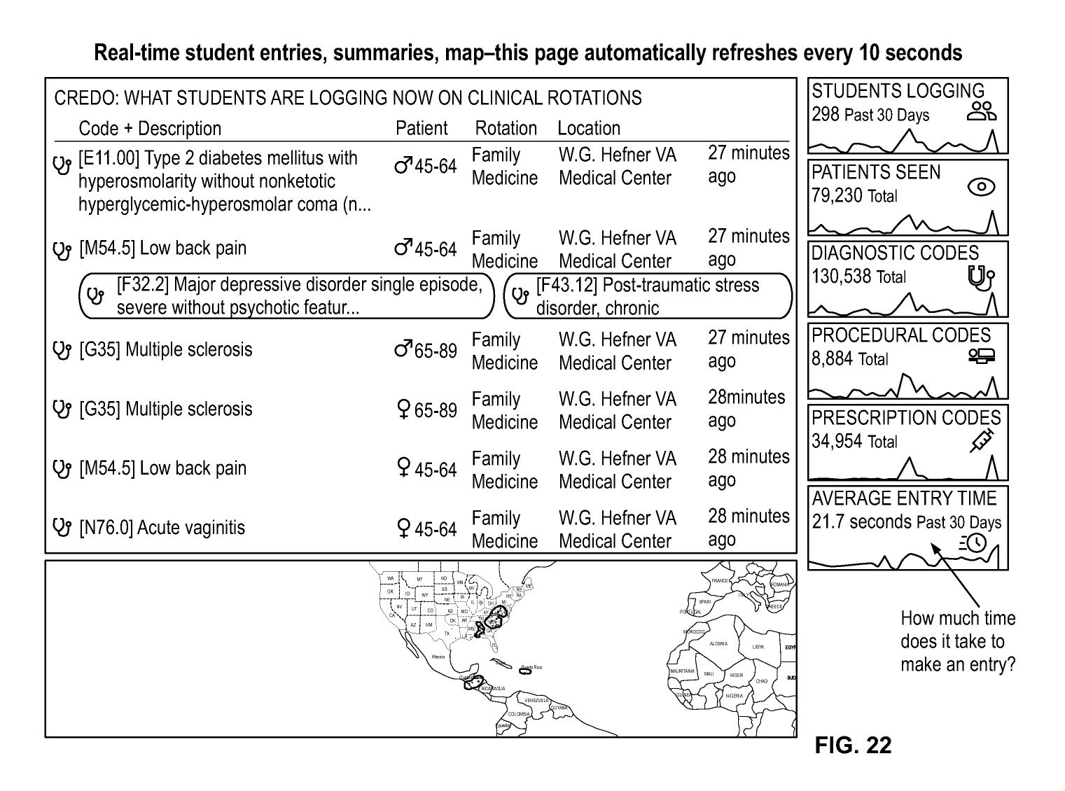

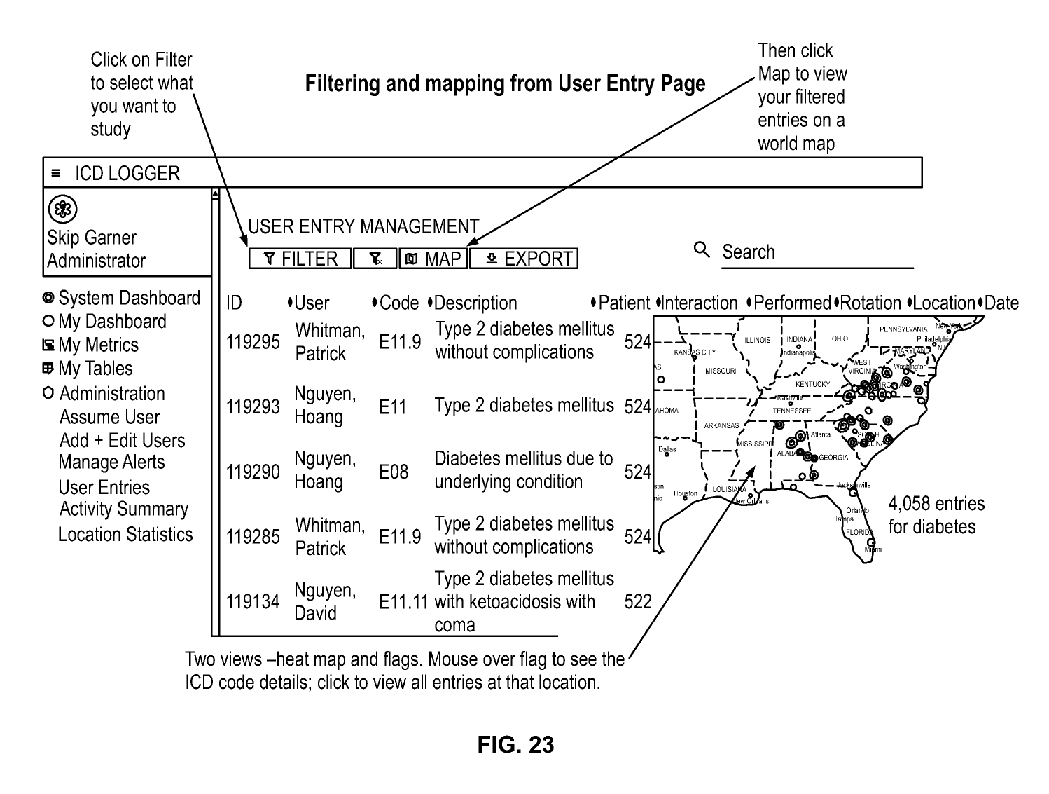

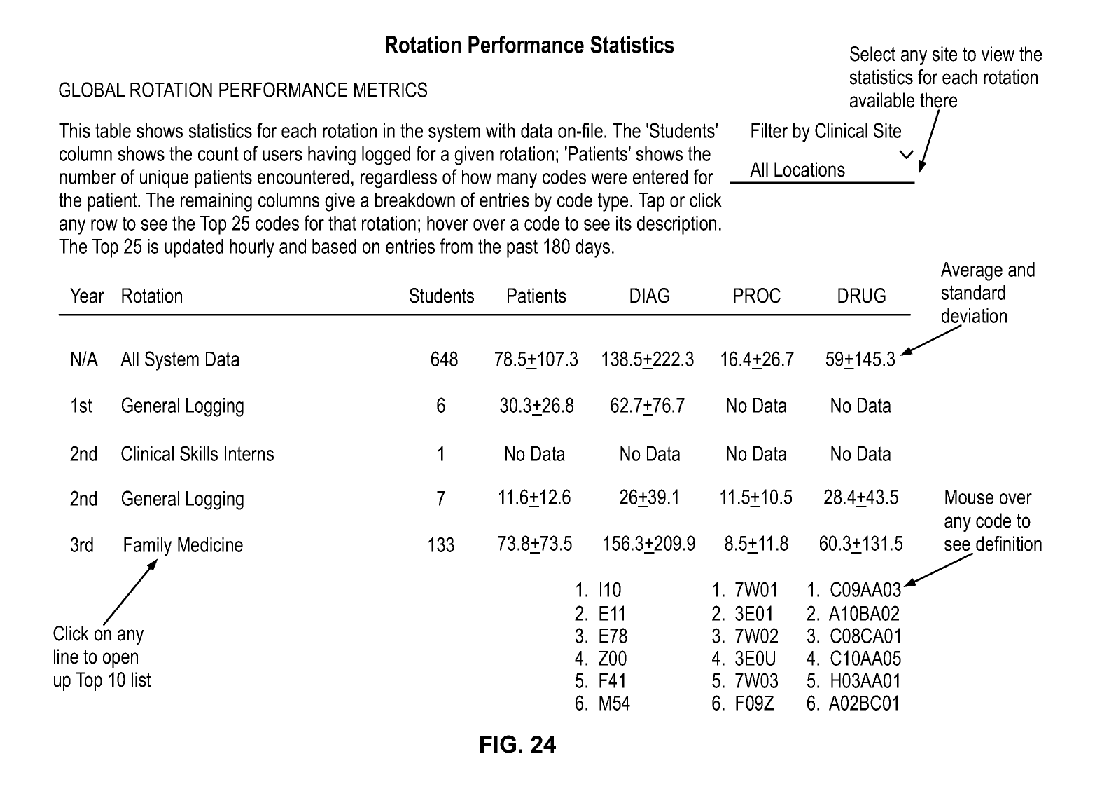

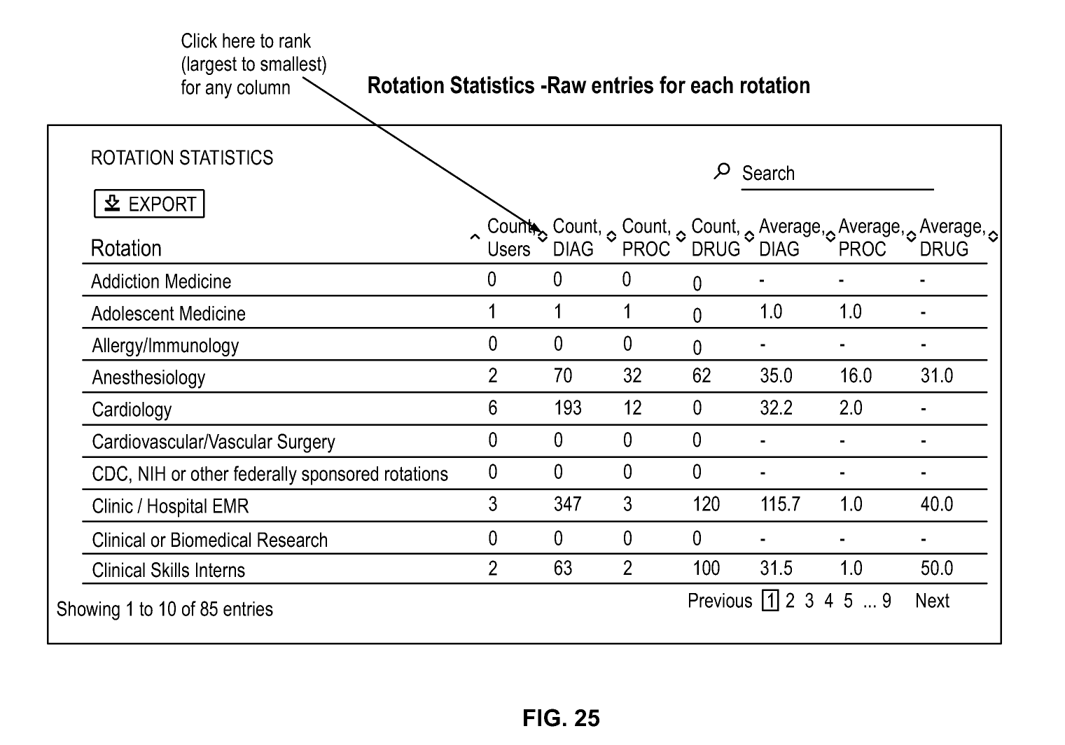

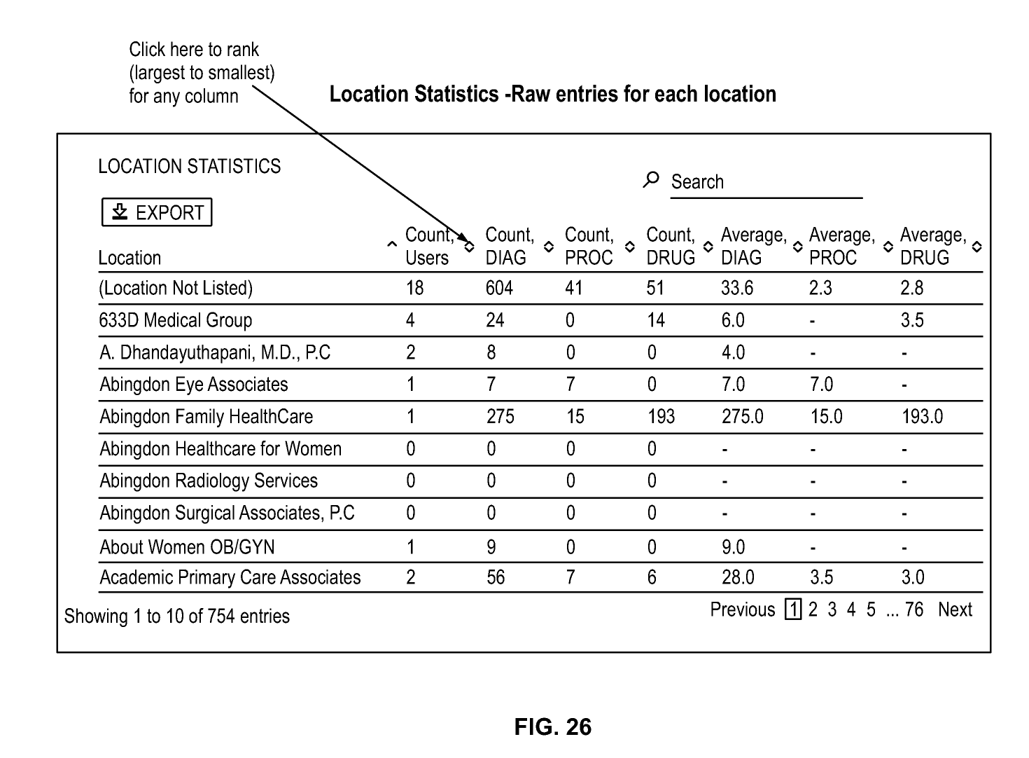

FIG. 21 shows an exemplary screen illustrating a time graph of students who are active. FIG. 22 shows an exemplary screen illustrating real-time student entries, summaries, map which may refresh every 10 seconds. FIG. 23 shows an exemplary screen illustrating filtering and mapping from a User Entry Page. FIG. 24 shows an exemplary screen illustrating rotation performance statistics. FIG. 25 shows an exemplary screen illustrating rotation statistics-raw entries for each location. FIG. 26 shows an exemplary screen illustrating location statistics-raw entries for each location.

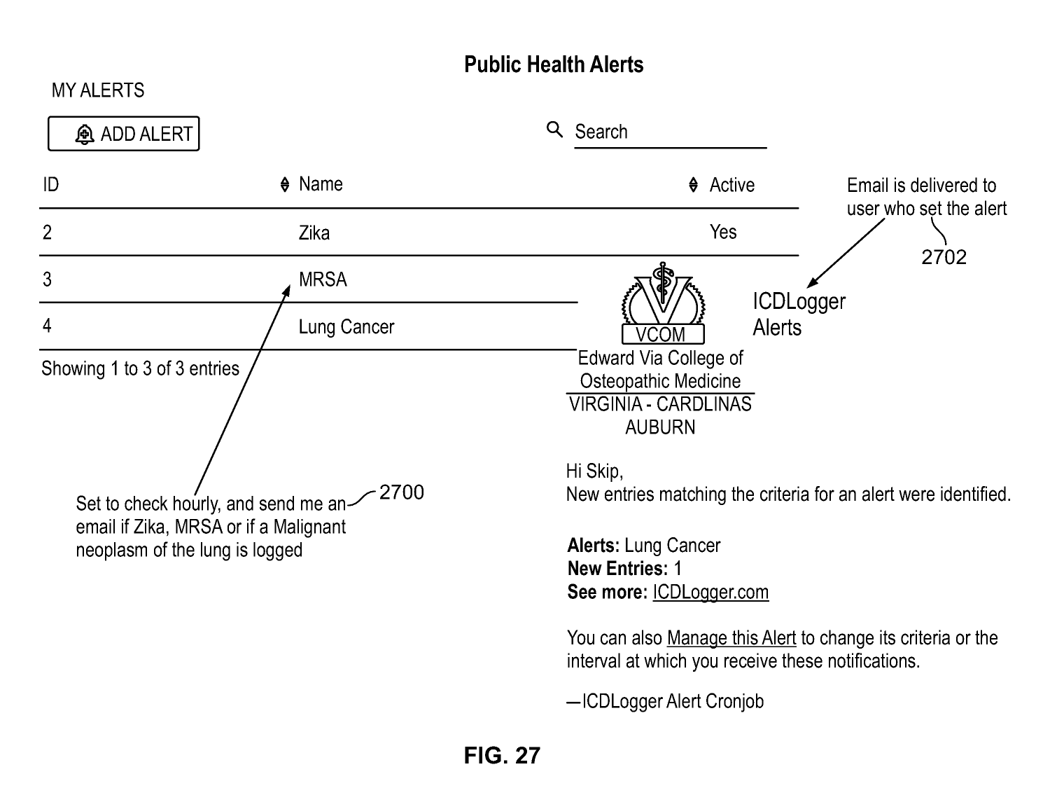

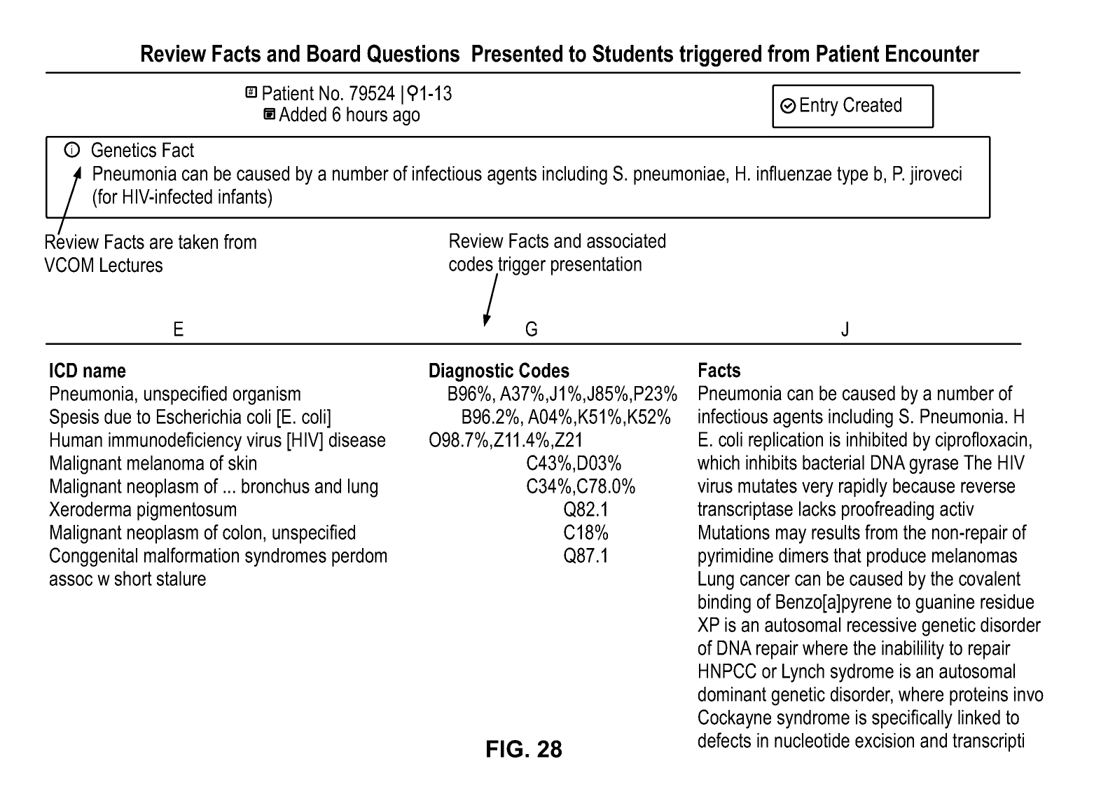

FIG. 27 shows an exemplary screen illustrating the utility of the present invention for issuing public health alerts. As shown, the system may be programmed to check hourly or for some other predetermined period of time for a known health hazard 2700. An email or some other form of alert such as a text message may be sent to a predetermined user or users 2702. FIG. 28 shows an exemplary screen illustrating review facts and board questions presented to students triggered from a patient encounter.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A system implemented on a distributed computer network for capturing experiences of healthcare trainees, the system comprising:
a plurality of electronic input devices, each of said input devices having an interface for interacting with a user including an input screen having data input fields, selection fields and activation buttons and output screens;
one or more back-end databases; and
said input devices in communication with said one or more back-end databases, said database configured to store input data from said input devices and to provide output data to said input devices.

2. The system of claim 1 further including a login system including user identification routines to establish user identity and user system access status.

3. The system of claim 1 further including a common language to identify and capture clinical or training encounters by healthcare trainees, said encounters included in said input data provided to said one or more back-end databases.

4. The system of claim 3 wherein said common language is ICD codes.

5. The system of claim 4 wherein said ICD codes provide a common medical vocabulary that captures diagnoses, procedures, and drugs during patient encounters.

6. The system of claim 5 further configured to use said captured codes to provide predetermined learning objectives, medical information or medical board review questions to a healthcare trainee.

7. The system of claim 5 further configured to provide real-time interactive information to a healthcare trainee associated with an inputted ICD code.

8. The system of claim 5 further configured to include a collection of databases containing annotated ICD codes to connect patient encounters, learning materials, review materials, and performance metrics.

9. The system of claim 5 further configured to permit healthcare trainees to search ICD codes using hierarchical menus.

10. The system of claim 5 further configured to permit healthcare trainees to search ICD codes using keywords.

11. The system of claim 5 further configured to permit healthcare trainees to search ICD codes by presenting frequently entered ICD codes.

12. The system of claim 5 further configured to permit healthcare trainees to link data entries to a selected ICD code.

13. The system of claim 5 further configured to permit healthcare trainees, for each encountered patient, the ability to input: 1) the ICD code that describes their diagnosis and treatment procedure; 2) codes for WHO listed and/or FDA approved drugs; 3) the patient gender and age; 4) free-text notes; 5) the clinic/hospital and rotation type as entered by the user and whether the training environment is a hospital or ambulatory; 6) their precise location; and 7) date/time of entry.

14. The system of claim 5 further configured to include a personally identifiable information detection system, said detection system prevents entry of personally identifiable information.

15. The system of claim 5 wherein said back-end database is configured to generate reports that enable the tracking and editing of data entries.

16. The system of claim 5 further configured to allow administrators to monitor the cumulative entries from all trainees.

17. The system of claim 5 further configured to monitor trends and trigger alerts from defined thresholds.

18. The system of claim 5 further configured to include displays adapted to monitor current activities being logged and to provide statistics that track and graph system-wide usage.

19. The system of claim 5 further configured to continuously process and provide data on a plurality of displays.

20. The system of claim 5 further configured to provide a healthcare trainee an interactive summary page of one or more of the entries entered by the trainee, said one or more entries are editable, searchable, and have a variety of ranked column views.

21. The system of claim 20 wherein said entries are associated with predetermined learning objectives or entries have predetermined codes that qualify for fulfillment.

22. The system of claim 20 wherein learning objectives or log entries are fulfilled by a manual entry by a trainee with a justification that is comprised of a reference, a reading, video or a lecture.

23. The system of claim 5 further configured to provide preceptors, faculty or administrators the ability to evaluate a trainee via a check off and text entry table.

24. The system of claim 5 further configured to provide preceptors, faculty and administrators access to trainee log entries, summaries and learning objective and log fulfillment tables to aid in the evaluation of a trainee.

25. The system of claim 5 further configured to allow evaluations to be conducted via a secure one-time link to the evaluation form.

26. The system of claim 25 further configured to automatically send emails to preceptors, faculty, and administrators with a link to initiate the evaluation process.

27. The system of claim 25 further configured to send reminders to evaluators if they have not submitted their evaluation by a set time.

28. The system of claim 5 further configured to allow an administrator to track an evaluator's completion of one or more evaluations.

29. The system of claim 5 further configured to capture evaluation information to determine if a trainee passes or fails a rotation.

30. The system of claim 5 wherein individual clinical training sites or preceptor performance or uniformity can be evaluated by the quantitative and qualitative performance of their trainees via the trainee's entries.

31. The system of claim 5 further configured to compute the average and standard deviation of trainee entries to determine if one or more trainees, preceptors or sites are significantly above or below average.

32. The system of claim 1 wherein review facts are presented to trainees for review following an entry of a given diagnostic, procedure or drug code.

33. The system of claim 32 wherein one or more facts are annotated by one or more codes to trigger the presentation of the one or more facts to a trainee.

34. The system of claim 1 wherein review questions and answers are presented to trainees for review following an entry of a given diagnostic, procedure or drug code.

35. The system of claim 34 wherein one or more facts are annotated by one or more codes to trigger the presentation of the one or more facts to a trainee.

36. The system of claim 1 wherein a response by a trainee to review questions is captured as either a right or wrong answer.

37. The system of claim 36 wherein a wrong answer is represented at a later time.

38. The system of claim 1 wherein associations between sites, preceptors, and students for each clinical rotation are imported, generated, or maintained.

39. The system of claim 1 further configured to allow a trainee to record their research experiences by entering titles, abstracts, manuscripts, data or other information regarding research activities.

40. The system of claim 1 further configured to allow faculty to enter their research interests and capabilities and desire to accept a student to do research so that students can search for and identify research mentors.

41. The system of claim 1 further configured to allow a trainee to enter their research interests and capabilities for comparison with faculty research interests and capabilities to identify matches.

42. The system of claim 3 wherein said common language is ICD code descriptions.

43. The system of claim 3 wherein said common language is CPT codes.

44. The system of claim 3 wherein said common language is CPT code descriptions.

45. The system of claim 3 wherein said common language is comprised of billing codes.

46. The system of claim 5 further configured to include a personally identifiable information detection system, said detection system prevents entry of personally identifiable information by searching for first names, last names, and patterns that resemble phone numbers, social security numbers.

47. The system of claim 5 further configured to monitor trends and trigger alerts from defined thresholds, said alerts comprising email, text or call to a person or another system.
